(19) United States
(12) Patent Application Publication
(10) Pub. No.: US 2025/0000442 A1
(43) Pub. Date: Jan. 2, 2025

(54) METHOD AND APPARATUS FOR DETERMINING PATELLAR DISLOCATION

(71) Applicant: ELEVENLITER INC., Seoul (KR)

(72) Inventors: Kwang Hyeon KIM, Seoul (KR); Ju Ho JUNG, Yongin-si (KR); Ju Yong PARK, Seoul (KR); Geon Woo PARK, Daejeon (KR)

(73) Assignee: ELEVENLITER INC., Seoul (KR)

(21) Appl. No.: 18/523,634

(22) Filed: Nov. 29, 2023

(30) Foreign Application Priority Data
Jun. 28, 2023 (KR) 10-2023-0083403
Jun. 28, 2023 (KR) 10-2023-0083413
Oct. 30, 2023 (KR) 10-2023-0146822

(51) Int. Cl. A61B 5/00; A61B 5/107; G06T 7/00; G06V 10/764

(52) U.S. Cl. CPC A61B 5/4585; A61B 5/1071; G06T 7/0012; G06V 10/764; A61B 2503/40

(57) ABSTRACT

The present disclosure relates to a method and apparatus for determining disease in pets, and more specifically, to a method of determining patellar dislocation based on AI and an apparatus to which the method is applied. A method of operating an apparatus for determining patellar dislocation according to an embodiment of the present invention includes an operation of obtaining a back view image including a hind leg area of the pet, an operation of outputting information on patellar dislocation in the pet using the back view image as an input to a pre-trained patellar dislocation prediction model, in which the patellar dislocation prediction model includes an operation of outputting first dislocation information of the pet when the back view image is a first back view image including first diagnostic angle information related to a patella of the pet and an operation of outputting second dislocation information of the pet when the back view image is a second back view image including information on a second diagnostic angle that is a different angle from the first diagnostic angle.

### BACKGROUND

### 1. Field of the Invention

The present disclosure relates to method and apparatus for determining a disease in pets, and more particularly, to a method and apparatus for determining patellar dislocation that can determine patellar dislocation, which is a specific disease in a pet, from a photo image of the pet using an artificial intelligence model.

### 2. Discussion of Related Art

Pets are a general term for animals that live with people, and representative examples of pets may include dogs and cats. Recently, as the number of people raising pets has increased and people's awareness of their pets' health has improved, industries related to the pets are also growing rapidly.

In particular, since pets cannot speak like humans, health care of pets requires special attention and observation. Accordingly, interest in pet care is also increasing, and technologies to manage pet health through technological approaches are in the spotlight. Representative pet care includes patellar dislocation, dental disease, obesity management, etc., that frequently occur in pets, and such pet care can be performed in various forms and methods.

One of the most common diseases that may occur in pets is patellar dislocation. Patella refers to a small, pebble-shaped concave bone in a middle of a front knee, which enables a knee to bend and straighten, and serves to protect a knee joint. The state in which the patella deviates from its original position is called patellar dislocation, and the patellar dislocation occurs particularly frequently in small dogs.

Generally, the patellar dislocation is diagnosed by a veterinarian in a hospital by palpating the knee joint at various angles and directions, or diagnosed by reading medical imaging. Since the patellar dislocation cannot be identified with the naked eye, in order to diagnose whether the patellar dislocation occurs, pet's guardians are required to visit a veterinary hospital. When the patellar dislocation occurs, it may vary depending on the progress level of the dislocation, but in general, the dislocation makes normal walking difficult. In addition, once the dislocation begins, the dislocation recurs repeatedly. As a result, prevention and early diagnosis of patellar dislocation are required.

However, in the case of the patellar dislocation, there is a problem in that once pets become accustomed to the dislocated state, the pets do not feel much pain, and since pets cannot express themselves, it is difficult to immediately determine whether the patellar dislocation has occurred unless the pet's guardians observe carefully.

For the above reasons, the pet's guardians often visit veterinary hospitals after the patellar dislocation progresses to a more severe level. In this case, various additional problems may occur, such as poor prognosis, long-term treatment time, or high treatment costs.

Recently, to solve these problems, joint diagnosis assistance technology based on artificial intelligence technology has emerged. Examples of the joint diagnosis assistance technology may include a technology of predicting and diagnosing a joint disease using an image classification artificial intelligence model from photo images of specific parts of a pet, and the like. Using the artificial intelligence technology, the pet's guardians may capture specific parts of a pet using only a smartphone and has a joint disease diagnosed based on AI technology without visiting a veterinary hospital, so they may recognize the occurrence of the joint disease at an early stage.

However, most of these diagnostic technologies cannot diagnose the presence or absence of dislocation by targeting the patella, which is a specific joint, among various joints. Although these techniques diagnose that the patellar dislocation occurs, these diagnostic technologies use X-ray photographs instead of using simple photo images or derive low-accuracy diagnostic results due to noise caused by the tail/fur, an inaccurate keypoint extraction, etc.

Therefore, there is a need for a technology that can infer the presence or absence of patellar dislocation with high accuracy using photo images of hind legs of a targeted pet.

### RELATED ART DOCUMENT

### Patent Document

(Patent Document 0001) Korean Patent No. 10-2415305

(Patent Document 0002) Korean Patent No. 10-2045741

(Patent Document 0003) Korean Patent No. 10-2304088

(Patent Document 0004) Korean Patent Laid-Open Publication No. 2021-0108686

(Patent Document 1) Korean Patent No. 10-2255483

(Patent Document 0006) Korean Patent No. 10-2021-0102721

(Patent Document 0007) Korean Patent No. 10-1759203

(Patent Document 0008) U.S. Pat. No. 10,477,180

(Patent Document 0009) Korean Patent Laid-Open Publication No. 10-2021-0107115

(Patent Document 0010) Korean Patent No. 10-2218752

### Non-Patent Document

(Non-Patent Document 0001) Jong-mo Lee, Ji-in Kim, Hyeong-seok Kim, “Veterinary Patellar Deviation Diagnosis System with Deep-Learning,” PROCEEDINGS OF HCI KOREA 2021, Conference Presentation Collection (January, 2021): 475-480

(Non-Patent Document 0002) Young-seon Han, In-cheol In, and Won-du Jang, “Recognition of Tooth and Tartar Using Artificial Neural Network,” Master of Engineering Thesis (February, 2022)

### SUMMARY OF THE INVENTION

The present disclosure provides a method and apparatus for determining patellar dislocation that are capable of outputting dislocation information with high accuracy by calculating a diagnostic angle from one back view photo image of a pet.

In addition, the present disclosure provides a method and apparatus for determining patellar dislocation that are capable of determining the presence or absence of patellar dislocation with high accuracy by removing a specific body area through performing pre-processing on a back view image of a pet.

Objects of the present specification are not limited to the above-described objects, and other objects and advantages of the present specification that are not described may be understood by the following description and will be more clearly appreciated by exemplary embodiments of the present specification. In addition, it may be easily appreciated that objects and advantages of the present specification may be realized by means described in the claims and a combination thereof.

According to an embodiment of the present disclosure, a method of operating an apparatus for determining patellar dislocation that determines a joint disease in a pet includes an operation of obtaining a back view image including a hind leg area of the pet and an operation of outputting information on patellar dislocation in the pet using the back view image as an input to a pre-trained patellar dislocation prediction model, in which the patellar dislocation prediction model includes an operation of outputting first dislocation information of the pet when the back view image is a first back view image including first diagnostic angle information related to a patella of the pet and an operation of outputting second dislocation information of the pet when the back view image is a second back view image including information on a second diagnostic angle that is a different angle from the first diagnostic angle.

The patellar dislocation prediction model may include an image classification model that outputs a feature vector from the back view image, and an object detection model that outputs a keypoint from the back view image.

The patellar dislocation prediction model may further include a classifier that classifies the back view image based on the feature vector and the keypoint and outputs dislocation information of the pet.

The dislocation information may include at least one of a probability of presence of dislocation, a degree of dislocation progression, and a dislocation position visualization image.

The object detection model may calculate a diagnostic angle for diagnosing the patellar dislocation based on the keypoint.

The keypoint may include a first keypoint, a second keypoint, and a third keypoint, and the first keypoint may correspond to an innermost point of a sole of one side of the pet, the second keypoint may correspond to an outermost point of a knee of one side of the pet, and the third keypoint may correspond to an uppermost point among points where a body of the pet intersects a line perpendicular to a ground and passing through the first point.

The diagnostic angle may be a Q angle calculated by a sum of a first angle formed by a first straight line that passes through the second keypoint and is perpendicular to the ground and a second straight line that passes through the first keypoint and the second keypoint and a second angle formed by the first straight line and a third straight line that passes through the second key point and the third key point.

The keypoint may be generated at left, right, or both sides of hind legs of the pet in the back view image.

When the keypoint is generated at both sides of hind legs of the pet, the classifier may output the dislocation information of the pet using a Q angle of one hind leg having a wider Q angle among Q angles calculated for each of the both sides of hind legs of the pet as an input.

The method may further include, before outputting the information on the patellar dislocation in the pet, an operation of performing pre-processing on the back view image.

The operation of performing the pre-processing may include an operation of identifying a specific body area of the pet included in the back view image using an image classification model and an operation of removing a part or all of the identified specific body area from the back view image.

The operation of performing the pre-processing may further include an operation of performing the pre-processing based on a breed of the pet, remove the specific body area of the pet depending on whether the hind leg area is covered in the back view image when the pet included in the back view image is a first breed, and may not remove the specific body area when the pet included in the back view image is a second breed.

The operation of performing the pre-processing may further include an operation of outputting estimated keypoints from the back view image using the object detection model, compare a position of at least one of the estimated keypoints with a position of the specific body area of the identified pet, and determine that the hind leg area is covered to remove the specific body area of the pet when the position of the specific body area corresponds to a position of at least one of the estimated keypoints.

According to another embodiment of the present disclosure, an apparatus for determining patellar dislocation include a memory, at least one processor that executes instructions stored in the memory, in which the processor acquires a back view image including a hind leg area of a pet, and outputs information on patellar dislocation in the pet using the back view image as an input to a pre-trained patellar dislocation prediction model, the patellar dislocation prediction model outputs first dislocation information of the pet when the back view image is a first back view image including first diagnostic angle information related to a patella of the pet, and outputs second dislocation information of the pet when the back view image is a second back view image including information on a second diagnostic angle that is a different angle from the first diagnostic angle.

The patellar dislocation prediction model may include an image classification model that outputs a feature vector from the back view image, and an object detection model that outputs a keypoint from the back view image.

The patellar dislocation prediction model may further include a classifier that classifies the back view image based on the feature vector and the keypoint and outputs the dislocation information of the pet.

Before outputting the information on the patellar dislocation in the pet, the processor may perform pre-processing on the back view image.

The processor may identify a specific body area of the pet included in the back view image using an image classification model, and remove a part or all of the identified specific body area from the back view image.

The processor may perform the pre-processing based on a breed of the pet, and remove the specific body area of the pet depending on whether the hind leg area is covered in the back view image when the pet included in the back view image is a first breed, and may not remove the specific body area when the pet included in the back view image is a second breed.

The processor may output an estimated keypoint from the back view image using the object detection model, and compare a position of at least one of the estimated keypoints with a position of a specific body area of the identified pet, and determine that the hind leg area is covered to remove the specific body area of the pet when the position of the specific body area corresponds to the position of at least one of the estimated keypoints.

### BRIEF DESCRIPTION OF THE DRAWINGS

FIG. 1 is a block diagram illustrating an apparatus for determining a disease in a pet according to an embodiment of the present disclosure.

FIG. 2 is a configuration diagram schematically illustrating a configuration of a system including the apparatus for determining patellar dislocation in a pet according to the embodiment of the present disclosure.

FIG. 3 is a block diagram illustrating the configuration of the apparatus for determining patellar dislocation in a pet according to the embodiment of the present disclosure.

FIG. 4 is a reference diagram for describing keypoints recognized in a back view image of a pet by a point recognition unit according to an embodiment of the present disclosure.

FIG. 5 is a reference diagram for describing that a diagnostic unit according to an embodiment of the present disclosure calculates a diagnostic angle that is a criterion for diagnosing the patellar dislocation in the pet.

FIG. 6 is a reference diagram for describing an example in which the apparatus for determining patellar dislocation according to the embodiment of the present disclosure calculates bilateral diagnostic angles.

FIG. 7 is a reference diagram for describing an example in which the apparatus for determining patellar dislocation according to the embodiment of the present disclosure corrects a diagnostic angle.

FIG. 8 is an example of a capturing guideline provided to a user terminal by a guide unit according to an embodiment of the present disclosure.

FIG. 9 is a flowchart illustrating a method of determining patellar dislocation in a pet according to an embodiment of the present disclosure.

FIGS. 10 and 11 are reference diagrams for determining accuracy of image-based patellar dislocation diagnosis results according to an embodiment of the present disclosure.

FIG. 12 is a block diagram illustrating a configuration of an apparatus for determining a disease in a pet according to another embodiment of the present disclosure.

FIG. 13 is a flowchart illustrating a method of determining patellar dislocation in a pet according to another embodiment of the present disclosure.

FIG. 14 is a diagram for describing a first artificial neural network architecture of a patellar dislocation prediction model according to an embodiment of the present disclosure.

FIG. 15 is a diagram for describing a Q angle according to an embodiment of the present disclosure.

FIG. 16 is a diagram for describing a second artificial neural network architecture of the patellar dislocation prediction model according to an embodiment of the present disclosure.

FIG. 17 is a diagram for describing a third artificial neural network architecture of the patellar dislocation prediction model according to an embodiment of the present disclosure.

FIG. 18 is a diagram for describing a fourth artificial neural network architecture of the patellar dislocation prediction model according to an embodiment of the present disclosure.

FIG. 19 is a diagram for describing a fifth artificial neural network architecture of the patellar dislocation prediction model according to an embodiment of the present disclosure.

FIG. 20 is a diagram for describing a sixth artificial neural network architecture of the patellar dislocation prediction model according to an embodiment of the present disclosure.

FIG. 21 is a diagram for describing a seventh artificial neural network architecture of the patellar dislocation prediction model according to an embodiment of the present disclosure.

FIG. 22 is a diagram for describing an eighth artificial neural network architecture of the patellar dislocation prediction model according to an embodiment of the present disclosure.

FIG. 23 is a diagram for describing a ninth artificial neural network architecture of the patellar dislocation prediction model according to an embodiment of the present disclosure.

FIGS. 24A to 24C are diagrams illustrating a user interface screen on a user terminal according to input of back view images of different pets in an embodiment of the present disclosure.

FIGS. 25A and 25B are diagrams illustrating dislocation information on the user terminal according to the input of the back view images of different pets in the embodiment of the present disclosure.

FIG. 26 is a diagram illustrating dislocation information on each back view image of different breeds of pets having the same diagnostic angle information in the embodiment of the present disclosure.

FIG. 27 is a diagram illustrating dislocation information on each back view image of the same breed of pets having different diagnostic angle information in another embodiment of the present disclosure.

FIGS. 28A and 28B are diagrams illustrating an additional information screen provided to the user terminal according to the dislocation information in an embodiment of the present disclosure.

FIGS. 29A to 29C are diagrams illustrating the additional information screen provided to the user terminal according to the dislocation information in the embodiment of the present disclosure.

FIG. 30 is a flowchart illustrating a pre-processing method that is a method using a tail removal module in an embodiment of the present disclosure.

FIG. 31 is a flowchart illustrating the pre-processing method that is a method using a tail removal module in the embodiment of the present disclosure.

FIGS. 32A and 32B are user terminal screens illustrating back view images of first and second breeds in an embodiment of the present disclosure.

FIGS. 33A to 33C are diagrams illustrating a tail pre-processing operation process of a back view image in an embodiment of the present disclosure.

FIGS. 34A and 34B are diagrams illustrating back view images of a pet before and after pre-processing in an embodiment of the present disclosure.

FIG. 35 is a hardware configuration diagram of a computing system described in an embodiment of the present disclosure.

FIG. 36 is a configuration diagram of a system for analysis of obesity in a pet according to an embodiment of the present disclosure.

FIG. 37 is a flowchart of a method of analysis of obesity in a pet according to some embodiments of the present disclosure.

FIGS. 38 and 39 are flowcharts for describing in more detail the method of analysis of obesity in a pet described with reference to FIG. 2.

FIG. 40 is a diagram for describing a method of determining obesity in a pet used in some embodiments of the present disclosure.

FIG. 41 is a diagram illustrating a method of minimizing body outline flatness of a pet used in some embodiments of the present disclosure.

FIG. 42 is a diagram illustrating photo data of a pet whose body outline flatness is greater than or equal to a predefined reference used in some embodiments of the present disclosure.

FIGS. 43 and 44 are diagrams illustrating screens suggesting guiding a pet through a specific operation provided to a user when photo data needs to be reacquired, which may be implemented in some embodiments of the present disclosure.

FIG. 45 is a diagram for describing the photo data of the pet whose body outline flatness is greater than or equal to the predefined reference used in some embodiments of the present disclosure.

FIGS. 46 to 49 are diagrams illustrating screens suggesting moving a capturing device provided to the user when the photo data, which may be implemented in some embodiments of the present disclosure, needs to be reacquired.

FIG. 50 is a hardware configuration diagram of a computing device to which a program of analysis of obesity of a pet may be applied in another embodiment of the present disclosure.

FIG. 51 is a configuration diagram of a system for analysis of oral information in a pet according to an embodiment of the present disclosure.

FIG. 52 is a flowchart of a method of analysis of oral information in a pet according to some embodiments of the present disclosure.

FIG. 53 is a diagram illustrating a convolutional neural network (CNN) that derives information on color uniformity of a crown area used in some embodiments of the present disclosure.

FIG. 54 is a diagram for describing the CNN that derives the information on the color uniformity of a gingival area used in some embodiments of the present disclosure.

FIG. 55 is a diagram for describing a process of feature fusion of two pieces of information described using FIGS. 3 and 4.

FIG. 56 is a diagram for describing a CNN that scores information on a degree of recession of a gum line.

FIG. 57 is a diagram for describing an artificial neural network architecture of a model used in some embodiments of the present disclosure.

FIG. 58 is a hardware configuration diagram of a computing system described in some embodiments of the present disclosure.

### DETAILED DESCRIPTION OF EXEMPLARY EMBODIMENTS

Hereinafter, various exemplary embodiments of the present disclosure will be described in detail with reference to the accompanying drawings. Various advantages and features of the present disclosure and methods of accomplishing them will become apparent from the following description of embodiments with reference to the accompanying drawings. However, the technical idea of the present disclosure is not limited to embodiments to be described below, but may be implemented in various different forms, only the following embodiments will be provided only in order to make the technical idea of the present disclosure complete and allow those skilled in the art to completely recognize the scope of the present disclosure, and the technical idea of the present disclosure will be defined by the scope of the claims.

In describing various embodiments of the present disclosure, well-known functions or constructions will not be described in detail since they may unnecessarily obscure the understanding of the present disclosure.

Unless otherwise defined, terms (including technical and scientific terms) used in the following embodiments may be used with meanings that may be commonly understood by those skilled in the art to which this disclosure pertains, but this may vary depending on the intention or precedent of engineers working in the related field, the emergence of new technology, etc. Terms used in the present disclosure are for describing embodiments rather than limiting the present disclosure.

Singular expressions used in the following embodiments include plural concepts, unless the context clearly specifies singularity. In addition, plural expressions include singular concepts unless the context clearly indicates otherwise.

In addition, terms such as “first,” “second,” “A,” “B,” “(a),” and “(b)” used in the following embodiments are only used to distinguish one component from another component, and the terms do not limit the nature, sequence, or order of the components in question.

Hereinafter, various embodiments of the present disclosure will be described in detail with reference to the accompanying drawings.

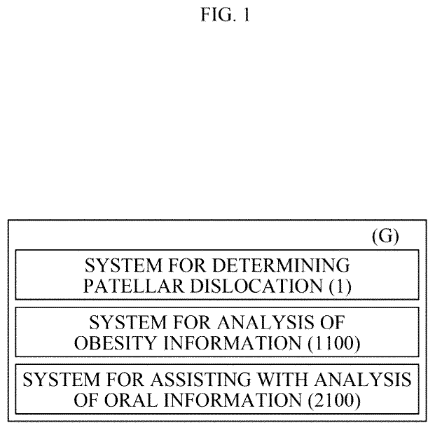

FIG. 1 is a block diagram illustrating an apparatus for determining a disease in a pet according to an embodiment of the present disclosure. In the present disclosure, an apparatus G for determining a disease in a pet is an apparatus for determining the occurrence of patellar dislocation, obesity, and oral diseases such as periodontitis/gingivitis, which are representative diseases frequently occurring in a pet, and as illustrated in FIG. 1, may include a system 1 for determining patellar dislocation, a system 1100 for analysis of obesity in a pet, and a system 2100 for assisting with analysis of oral information. Hereinafter, a method of operating a system for determining a disease for each disease will be described according to an embodiment of the present disclosure.
[1]

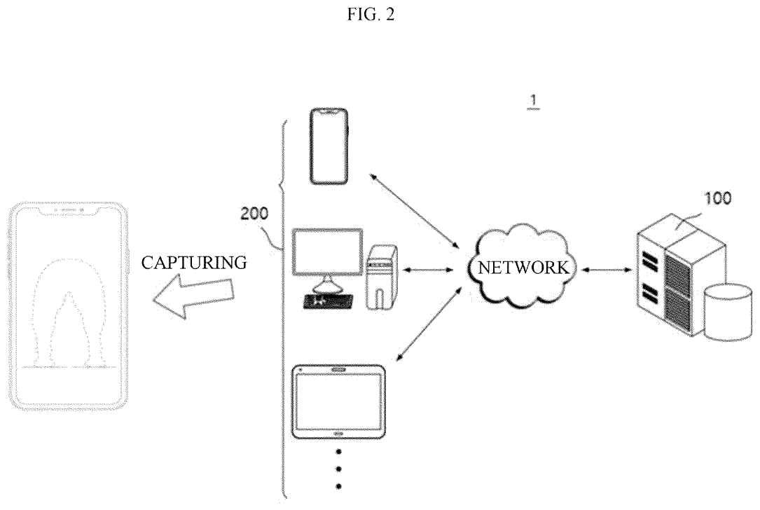

As illustrated in FIG. 2, a system 1 for determining patellar dislocation in a pet (hereinafter abbreviated as ‘system’) according to the present embodiment may include an apparatus 100 for determining patellar dislocation and a user terminal 200. A user of the user terminal 200 may capture a hind leg area of a targeted pet 10 so that the hind leg area is visible from the front, and transmit the captured image, which is the result of the capturing, to the apparatus 100 for determining patellar dislocation. The user terminal 200 may be equipped with a camera module for capturing a back image of a pet to directly acquire the back image of the pet. However, the user terminal 200 may receive the back image previously captured by an external camera module or transmit the back image stored in its own memory to the apparatus 100 for determining patellar dislocation. Here, the captured image and the back image refer to a back view image of a pet which is a digital photo captured from behind the pet so that buttocks and hind legs of the pet as the other side of a head area of the pet are visible.

The pet described in the present disclosure may include various types and breeds of pets that are likely to develop patellar dislocation disease. For example, the pet may be a dog that is a target animal with a high incidence of patellar dislocation disease. However, the target animal is not necessarily limited thereto, and includes all pets in which the patellar dislocation occurs and can be diagnosed.

The user terminal 200 is a user terminal device used by a user who wants to diagnose the presence and degree of patellar dislocation in a pet, and as the user terminal 200, a smartphone, a tablet PC, a laptop, a desktop, etc., may be used. The user terminal 200 is connected to the apparatus 100 for determining patellar dislocation through various known wired and wireless communication networks such as wireless fidelity (WiFi), long term evolution (LTE), and 5^{th} generation (5G), and transmits and receives various data necessary in the process of diagnosing a degree of patellar dislocation in a pet, including back images obtained by capturing the back of the pet, and diagnostic results.

The user terminal 200 may have a pet care service application installed therein, with the pet care service application being connected to the apparatus 100 for determining patellar dislocation or a separate service server (not illustrated), and the pet care service application may provide a photo capturing user interface (UI) that guides photo capturing so that the hind leg area of the pet is visible from the front. Of course, the user terminal 200 has a web browser installed therein, with the web browser accessing a front-end web page served by the apparatus 100 for determining patellar dislocation or the separate service server (not illustrated), and the front-end web page may provide a photo capturing UI that guides photo capturing so that the hind leg area of the pet is visible from the front.

The apparatus 100 for determining patellar dislocation may refer to a virtual machine or container provisioned on one or more cloud nodes. Of course, the apparatus 100 for determining patellar dislocation may be implemented through one or more on-premise physical servers. The apparatus 100 for determining patellar dislocation may store definition data of a patellar dislocation prediction model previously generated through machine learning. A machine learning runtime (ML runtime), which is software for executing the patellar dislocation prediction model, may be installed.

In other words, the apparatus 100 for determining patellar dislocation may receive a captured image of a hind leg of a pet from the user terminal 200, input data of the acquired captured image of the hind leg into the patellar dislocation prediction model, generate patellar dislocation-related analysis results using the data output from the patellar dislocation prediction model, and transmit the result data including the generated patellar dislocation-related analysis results to the user terminal 200.

In some embodiments, the apparatus 100 for determining patellar dislocation may perform machine learning on the patellar dislocation prediction model periodically or aperiodically and deploy the updated patellar dislocation prediction model to the user terminal 200. That is, in this case, the apparatus 100 for determining patellar dislocation may serve as a device that executes a machine learning pipeline of the patellar dislocation prediction model, and the user terminal 200 may serve as a type of edge device that executes the machine learning application.

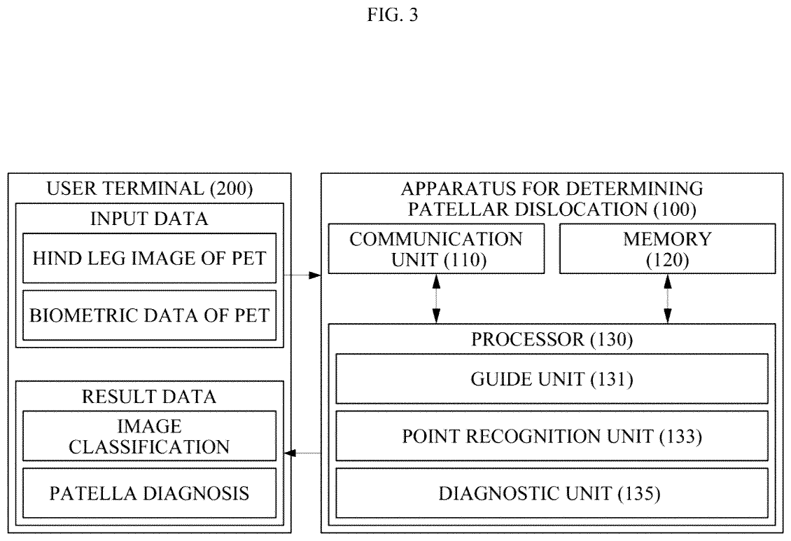

FIG. 3 is a block diagram illustrating the configuration of the apparatus for determining patellar dislocation in a pet according to the embodiment of the present disclosure. Hereinafter, the configuration of the apparatus for determining patellar dislocation in a pet will be described with reference to FIG. 3.

Referring to FIG. 3, the apparatus 100 for determining patellar dislocation includes a communication unit 110, a memory unit 120, and a processor 130.

The communication unit 110 is a module that performs communication with the user terminal 200, and receives a back view image of a pet, which is the back image of the pet, and biometric data of the targeted pet from the user terminal 200, provides diagnostic results of patellar dislocation based on the received back view image and biometric data, and transmits and receives various data necessary for the image capturing process and diagnosis process in the user terminal 200. The communication may be achieved through various known wired and wireless communication methods such as WI-FI, Ethernet, LTE, and 5G.

The memory unit 120 is implemented using memory elements such as random access memory (RAM), read-only memory (ROM), electrically erasable programmable read-only Memory (EEPROM), and flash memory, and may store various operating systems (OS), middleware, platforms, program codes for diagnosing patellar dislocation based on an image of a pet, and various applications.

In addition, as will be described below, the memory unit 120 may store reference information for determining the presence or absence and degree of patellar dislocation, correction information for correcting a diagnostic angle that is the basis for diagnosing the patellar dislocation, and an operation module or an artificial intelligence model that determines the presence or absence of patellar dislocation in a pet for the input image.

The processor 130 uses information stored in the memory unit 120 to process the back image of the pet received from the user terminal 200 and diagnose the presence or absence and degree of patellar dislocation in a pet in an image.

Referring to FIG. 3, the processor 130 includes a point recognition unit 133, a diagnostic unit 135, and a guide unit 131.

The point recognition unit 133 determines a plurality of keypoints that are the basis for diagnosing the patellar dislocation in the back image of the pet received through the user terminal 200. The plurality of keypoints become criteria for calculating the diagnostic angle for diagnosing the patellar dislocation. The keypoints are recognized for each of the left and right sides of the pet, and the keypoints recognized on one side are used to diagnose the patellar dislocation for the corresponding side. For example, the keypoint on the left side becomes a criterion for determining patellar dislocation of a left leg.

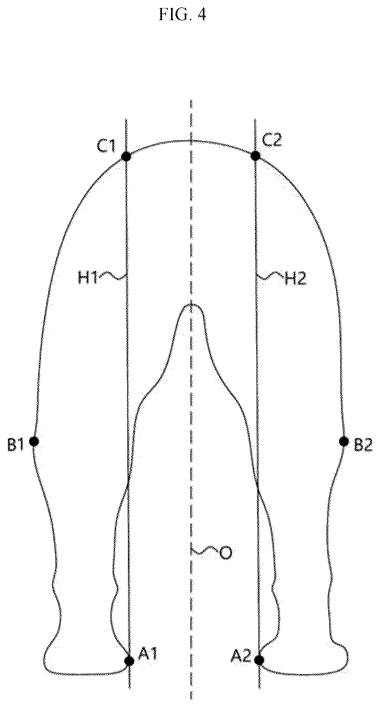

FIG. 4 is a reference diagram for describing keypoints determined by the point recognition unit 133 according to an embodiment of the present invention. For reference, based on a center line O that separates the left and right sides of the pet, a direction closer to the center line is defined as an inside, and a direction farther from the center line is defined as an outside.

Referring to FIG. 4, three keypoints are recognized on each of the left and right sides of the pet. First keypoints A1 and A2 are an innermost point of a sole of a pet, that is, points corresponding to the innermost point of the sole. Second keypoints B1 and B2 are outermost points of a knee of a pet, that is, points derived farthest from the knee to the outside. In addition, third keypoints C1 and C2 are points corresponding to uppermost points among points where a body of a pet intersects lines H1 and H2 perpendicular to the ground and passing through the first keypoints A1 and A2.

The point recognition unit 133 may recognize the keypoints based on a machine learning model trained to recognize three keypoints. The machine learning model may be generated based on various known machine learning/deep learning algorithms, such as a convolutional neural network (CNN) and a support vector machine (SVM), by applying, as training data, a back image of a pet, with each of the three keypoints on the left and right sides labeled.

In addition to the machine learning model, the point recognition unit 133 may also recognize feature points using known image processing algorithms for line identification, edge extraction, and corner point detection in images.

The machine learning model and image processing algorithm for keypoint recognition may be stored in the memory unit 120.

In addition, the point recognition unit 133 may automatically recognize keypoints through image processing, as described above, but may determine at least one of the first keypoint, the second keypoint, and the third keypoint based on the user input data received from the user terminal 200. For example, the point recognition unit 133 may receive a position of the first keypoint input from the user and determine the received position as the first keypoint. In this way, messages such as “Please click on the innermost point of the sole of the pet in the image.” and a sample image or picture of a pet on which the portion corresponding to the innermost point of the sole of the pet is displayed may be provided together so that a user may select the first keypoints A1 and A2 corresponding to the innermost points of the sole through a user interface of the user terminal 200.

Meanwhile, the point recognition unit 133 may receive a single image through the user terminal 200, but may also receive multiple images, recognize keypoints by selecting an image to be used for patella diagnosis from among the multiple images, or recognize keypoints for each of the multiple images. In this case, when the multiple images are used for diagnosis, the diagnostic results for each image may be synthesized to diagnose the patellar dislocation, for example, based on an average value of the diagnostic angles calculated from each image, etc.

The point recognition unit 133 may determine the quality of an image and select an image whose quality satisfies predetermined criteria as an image to be diagnosed. The image quality may be determined through the degree of blurring, specular highlight, dynamic range detection, etc. For reference, the image quality may be determined based on various known algorithms. For example, the degree of blurring may be quantified using a discrete wavelet transform, the amount of highlight due to reflection may be quantified by detecting hotspots using a threshold and/or Gaussian filter, and the dynamic range may be detected by calculating the distribution of colors available in each channel over the region of interest.

In addition, the point recognition unit 133 may recognize keypoints for multiple images, and select images to be used for diagnosis according to the recognition results of the keypoints. For example, images, in which keypoints cannot be recognized, may be excluded from diagnosis or, even if recognized, images where positions of corresponding keypoints on the left and right sides deviate from symmetrical positions by more than predetermined criteria, may be excluded from diagnosis.

The diagnostic unit 135 calculates a diagnostic angle for diagnosing the patellar dislocation based on the first keypoint, the second keypoint, and the third keypoint recognized through the point recognition unit 133, and determines the degree of the patellar dislocation based on the diagnostic angle.

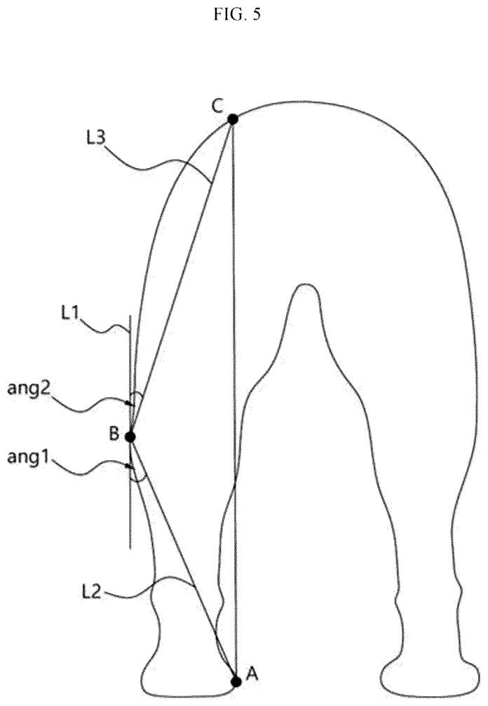

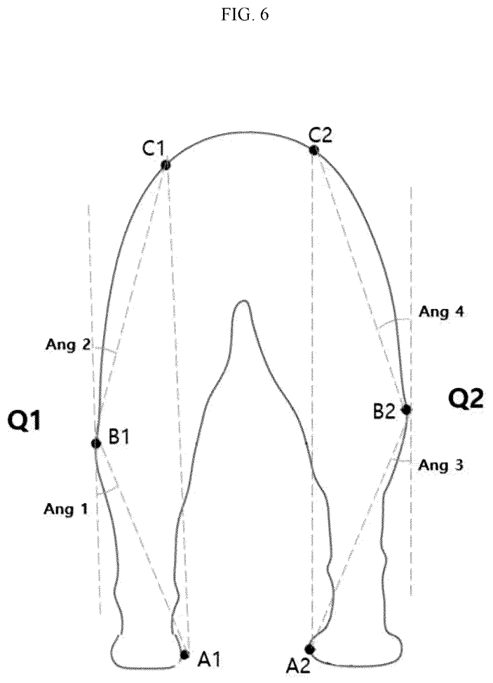

FIG. 5 is a reference diagram for describing that the apparatus for determining patellar dislocation according to an embodiment of the present invention calculates the diagnostic angle based on the keypoint, and FIG. 6 is a reference diagram for describing a process of calculating, by the apparatus for determining patellar dislocation according to an embodiment of the present invention, a diagnostic angle when generating keypoints on both sides of a hind leg. Hereinafter, a description will be provided with reference to FIGS. 5 and 6.

The diagnostic unit 135 calculates a first angle ang1 formed by a first straight line L1 perpendicular to the ground and passing through a second keypoint B and a second straight line L2 passing through a first keypoint A and the second keypoint B. In addition, a second angle ang2 formed by the first straight line L1 and a third straight line L3 passing through the second keypoint B and a third keypoint C is calculated.

Here, the diagnostic angle for determining patellar dislocation may be a Q angle calculated as the sum of the first angle ang1 and the second angle ang2.

For reference, as illustrated in FIG. 5, the diagnostic angle on the left side of the pet may be illustrated, but as illustrated in FIG. 6, the diagnostic angle on the right side may also be calculated in the same way as the left side.

Accordingly, the keypoint may be generated on one of the left or right hind leg of the pet in the back view image, or for both sides of the hind legs. When the keypoints are generated at both sides of the hind legs of a pet, the apparatus for determining patellar dislocation calculates the diagnostic angle for each of the hind legs of the pet, and determines whether the pet has patellar dislocation using a diagnostic angle of one hind leg, which has a wide diagnostic angle among the calculated diagnostic angles, as input.

For example, referring to FIG. 6, when the keypoints on both sides of the hind legs are identified through the point recognition unit 133, an angle of Q1, which is a diagnostic angle formed by a first keypoint A1, a second keypoint B1, and a third keypoint C1 of the identified left hind leg, is 30°, and an angle of Q2, which is a diagnostic angle formed by a first keypoint A2, a second keypoint B2, and a third keypoint C2 of the right hind leg, is 42°, the apparatus for determining patellar dislocation uses the larger angle Q2 to determine whether the pet has patellar dislocation.

In this way, the apparatus for determining patellar dislocation may calculate and compare the diagnostic angles on both sides of the hind legs of the pet, and determine the patellar dislocation based on the diagnostic angle of one hind leg, which has a large angle with a high incidence of patellar dislocation.

Therefore, by using the apparatus for determining patellar dislocation of the present disclosure, it is possible to not only secure high accuracy in determining the patellar dislocation, but also prevent the occurrence of patellar dislocation on the other side, which may be missed by calculating the diagnostic angle on only one side of the hind leg.

Subsequently, the memory unit 120 may store reference information regarding the diagnostic angle corresponding to the progress level of patellar dislocation divided according to the degree of progress of the patellar dislocation, and the diagnostic unit 135 may determine the progress level of patellar dislocation by comparing the diagnostic angle calculated through processing of the back image of the pet with the above referenced information.

The progress level of patellar dislocation may be divided into, stage 1, where slight dislocation occurs when artificial pressure is applied to patella, stage 2, where dislocation occurs when artificial pressure is applied to the patella, but returns naturally when the pressure is removed, stage 3, where dislocation occurs naturally without applying pressure to the patella and dislocation is less likely to return, and stage 4, where the patella remains dislocated and does not return.

The reference information regarding the diagnostic angle is a reference angle or a range of reference angle for determining each level corresponding to the above progression level. For example, the range of the reference angle corresponding to stage 2 of the patellar dislocation may be stored as 24.3° to 36.5°. As a result, when the diagnostic angle calculated from the image is calculated within the range of 24.3° to 36.5°, the patellar dislocation is determined to be stage 2. For reference, this is an example of a reference angle, and of course, it may be determined differently through experiment.

Meanwhile, when the back image of the pet is captured symmetrically, the diagnostic angle may be calculated accurately, but if the image is captured with the body of the pet angled to either the left or right side, when calculating the diagnostic angle using the image, the diagnostic results of the patellar dislocation may be inaccurate. To compensate for this, the diagnostic unit 135 may correct the diagnostic angle calculated based on the position difference between the corresponding keypoints on the left and right sides.

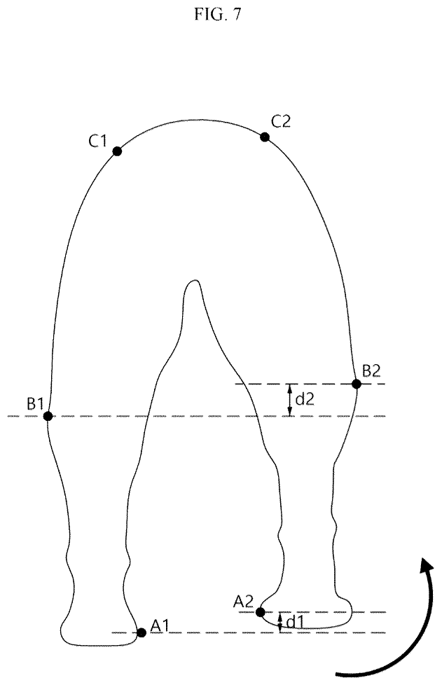

FIG. 7 is a reference diagram for describing an example in which the diagnostic unit 135 corrects a diagnostic angle according to an embodiment of the present invention.

FIG. 7 illustrates an example in which the keypoints are calculated for each of the left and right sides of the pet, when the left and right sides of the pet are not captured symmetrically and the back image is captured with the body of the pet angled to the right.

Referring to FIG. 7, it can be seen that, unlike what is illustrated in FIG. 4, the heights of the corresponding keypoints on the left and right sides do not appear approximately symmetrical, and the height difference appears. That is, the height difference between the first keypoint A1 on the left and the first keypoint A2 on the right is d1, and the height difference between the second keypoint B1 on the left and the second keypoint B2 on the right is d2, and the keypoints on the right side appear relatively higher than those on the left side.

The memory unit 120 may store correction information for correcting the diagnostic angle in response to a height difference d1 between the first keypoints on both sides recognized on the left and right sides of the pet, or a height difference d2 between the second keypoint on both sides, and the diagnostic unit 135 may correct the diagnostic angle calculated based on the stored correction information and diagnose the degree of patellar dislocation using the corrected diagnostic angle.

The correction information may be stored as specific correction values or correction coefficients for each value or range of d1, which is the height difference of the first keypoint, and d2, which is the height difference of the second keypoint. For example, just as d1 is ‘a’ cm to ‘b’ cm and d2 is ‘c’ cm to ‘d’ cm (a, b, c, d>0), as the form in which a specific correction angle value is subtracted from or added to the diagnostic angle primarily calculated for each range of d1 and d2 or a correction coefficient value multiplied by the diagnostic angle is stored, the correction values or correction coefficients may be stored as a data table defined according to the values or ranges of d1 and d2. In this case, only one of d1 and d2 may be used for correction, or a ratio value of d1 and d2 may be a criterion for determining the correction value or correction coefficient. In this way, when the ratio value of d1 and d2 is used for correction, each correction value or correction coefficient is stored corresponding to each ratio value or range of d1 and d2.

Meanwhile, the correction value or correction coefficient may be defined for only one of the first angle ang1 and the second angle ang2, or may be defined for each of the two angles ang1 and ang2, and may also be defined as the sum of the two angles ang1 and ang2. In this way, the specific correction value of the diagnostic angle based on the height difference between the keypoints on both sides may be determined through experiment.

As described above, the diagnostic unit 135 combines the results determined based on the diagnostic angle calculated from the back image of the pet and the determined results based on the biometric data of the pet received from the user terminal 200 to derive the final result regarding the degree of patellar dislocation in a pet.

Here, the biometric data of the pet may include at least one of age, breed, body condition score (BCS), clinical symptoms, past medical history, lifestyle, and living environment of a pet. For reference, the BCS is divided into 5-point or 9-point and is widely used in a veterinary field as an indicator that may determine the degree of obesity. The clinical symptoms, past medical history, lifestyle, and living environment may be determined through an online questionnaire through the user terminal 200.

The diagnostic unit 135 evaluates the risk of joint disease in pets based on the biometric data. Looking at an example of evaluating the risk of joint disease, as age increases, the risk may increase, and in the case of small dogs with breed predispositions to patella dislocation, such as Maltese, Chihuahua, and Poodle, the risk may increase. In addition, the BCS may be implemented to provide the determination reference information to the user terminal 200 and allow a user to directly input the determination reference information. When applying the 9-point BCS, the risk may increase from a BCS of 6 or higher. In addition, when the clinical symptoms related to joint disease, such as walking with one leg raised, appear, the risk increases and when there is a history of hospital visits due to joint disease in the past, the risk increases. Meanwhile, when pets are exposed to a living environment that is prone to joint disease, such as living in an indoor environment with slippery flooring or walking less than three times a week, the risk may increase.

In this way, the memory unit 120 may store evaluation criteria for evaluating the risk of joint disease for each biometric data item, and evaluate the risk of joint disease based on the evaluation criteria.

The diagnostic unit 135 may combine the image-based diagnosis results and the risk of joint disease based on the biometric data to derive the final result regarding the diagnosis of patellar dislocation. For example, when performing the determination based on the image, if the patellar dislocation is between stages 2 and 3 and the risk of joint disease based on the biometric data is high, the patellar dislocation may be finally determined to be stage 3.

Meanwhile, the diagnostic unit 135 may apply the back image of the pet received from the user terminal 200 to the learning model stored in the memory unit 120 to combine the output determination results of patellar dislocation and the diagnostic results based on the diagnostic angle calculated from the back image of the pet, thereby calculating the final result regarding the diagnosis of patellar dislocation.

Here, the learning model is a learning model that is trained to output the presence or absence of patellar dislocation in a pet in the input image by applying a back image of a normal pet without patellar dislocation and a back image of an abnormal pet with patellar dislocation as training data. Here, the learning may be achieved by supervised learning using a back image of a normal pet and a back image of an abnormal pet, each labeled with the absence or presence of patellar dislocation, as training data, or unsupervised learning based on unlabeled training data.

When generating the learning model, by applying a conditional deep convolutional generative adversarial network (cDCGAN) or the like to augment back image data of the normal and abnormal pets applied as the training data and apply the augmented data as the training data, problems of insufficiency and inconsistency of data may be compensated.

The diagnostic unit 135 may combine the determination results according to the above learning model and the determined results based on the diagnostic angle to derive the final result. For example, it is determined based on the diagnostic angle that the patellar dislocation is stage 1, but if it is assumed that the diagnostic angle is less than or equal to a predetermined reference value and very close to a normal range, when it is determined that there is no patellar dislocation according to the learning model, it may be determined to be normal despite the results based on the diagnostic angle, and conversely, when it is determined that there is the patellar dislocation according to the learning model, it may be determined that the patellar dislocation is stage 1.

The diagnostic unit 135 may combine a first diagnostic result based on the diagnostic angle, a second diagnostic result based on the learning model, and the risk of joint disease based on the biometric data to calculate the final diagnostic result regarding the presence or absence and the degree of patellar dislocation. In this way, it is possible to further improve the accuracy of diagnosis by combining multiple results. Meanwhile, when combining three results to derive the final diagnosis, different weights may be assigned to each result. For example, the greatest weight may be assigned to the first diagnostic result based on the diagnostic angle, and the smallest weight may be assigned to the risk of joint disease based on the biometric data.

Meanwhile, the weights of the three results may vary depending on the size of the specific diagnostic angle calculated from the back image. For example, when the calculated diagnostic angle is closer to the next level of patellar dislocation, a relatively smaller weight may be assigned to the diagnostic results based on the diagnostic angle than when the diagnostic angle is close to a middle value of the level.

In addition, the final diagnostic result may provide diagnostic accuracy along with the progress level of patellar dislocation. In addition, the accuracy of diagnosis may be determined by where the calculated diagnostic angle is located in the range of the progress level of patellar dislocation and comparing the risk of joint disease based on the biometric data and the image-based diagnosis results. For example, if the diagnostic angle is close to the boundary between progression levels, the accuracy may be relatively lower than when the diagnostic angle is close to the middle value of the range, and when the patellar dislocation is diagnosed as stage 3 based on the image but the risk is very low according to the biometric data, the accuracy may be calculated to below. The accuracy may be derived as ‘high’, ‘medium’, ‘low’ or as a percentage (%).

The final diagnostic result is provided to the user terminal 200 and displayed on the display of the terminal 200. The diagnostic result is stored in the memory unit 120 so that when the diagnosis is requested again for the pet in the future, the degree of progress of joint disease may be compared by comparing the diagnosis history.

Meanwhile, when the back image of the pet is captured through the user terminal 200, the guide unit 131 guides symmetrical capturing, horizontal capturing, and the like of the left and right sides of the pet so that an image with quality suitable for diagnosis may be captured, and when the captured image or uploaded image is not suitable for the patella diagnosis, the guide unit 131 guides the user terminal 200 to re-capture or re-upload an image.

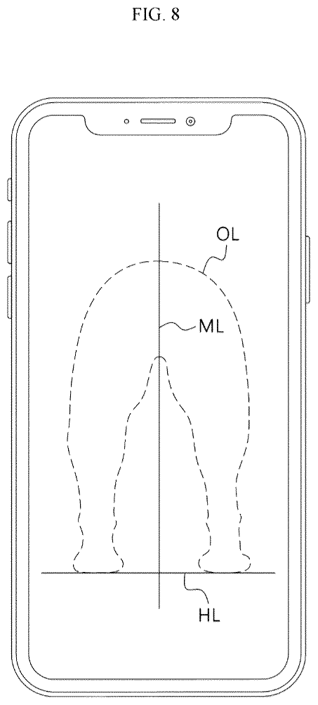

FIG. 8 illustrates an example of a capturing guideline provided by the guide unit 131 according to an embodiment of the present invention.

Referring to FIG. 8, the capturing guideline provided to the user terminal 200 to guide the capturing of the back image may include a midline ML to guide symmetrical capturing of the left and right sides with respect to an anus of a pet and a horizontal line HL to guide the level of both feet of the pet. A marker indicating the position of the anus may be included on the midline ML. Meanwhile, an outline line OL on the back of the pet may also be provided together as the capturing guideline. Since the outline line OL may vary depending on the size or breed of the pet, a plurality of outline lines are stored in advance in accordance with weight, size, breed, age, etc., and a plurality of outline lines may be selected and provided based on the biometric data of the pet received from the user terminal 200.

Meanwhile, the guide unit 131 may guide the user terminal 200 to re-capture or re-upload the image when the difference between positions of both sides of the keypoints recognized through the point recognition unit 133 differs by more than the predetermined reference value.

As illustrated in FIG. 7, the positions on both sides of at least one of the first keypoints A1 and A2, the second keypoints B1 and B2, and the third keypoints C1 and C2 identified on the left and right sides are compared individually, and when the height difference between the points on both sides is greater than or equal to the predetermined reference value, since errors may be large when calculating the diagnostic angle based on the corresponding image, the re-capturing or re-upload may be requested.

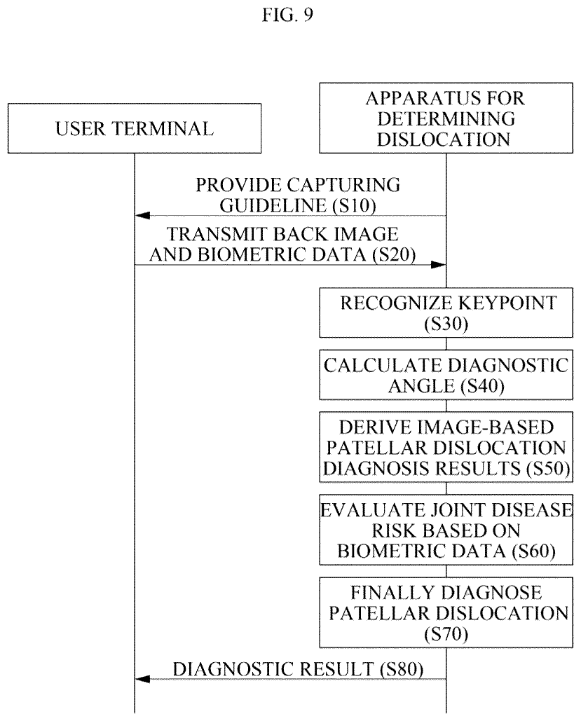

FIG. 9 is a flowchart illustrating a method of determining patellar dislocation in a pet according to an embodiment of the present disclosure. In order to avoid duplication of explanation, the contents described through the above embodiments will be omitted.

Referring to FIG. 9, the apparatus 100 for determining patellar dislocation generates a capturing guideline and provides the capturing guideline to the user terminal 200 (S10). As described above, the capturing guideline is intended to guide the symmetrical capturing and horizontal shooting on the left and right sides, and may include the midline ML, the horizontal line HL, the marker corresponding to the anus position, and the outline line OL of the pet. The capturing guideline is displayed on the display of the user terminal 200 to allow a user to perform the capturing according to the guideline, thereby improving the accuracy of diagnosis.

However, when the user terminal 200 does not perform the capturing and uploads an image that has already been captured or is captured in another apparatus, operation S10 may be omitted. Accordingly, the apparatus 100 for determining patellar dislocation may be implemented to provide the capturing guideline only when selected to perform the capturing by selecting whether to perform the capturing through the user terminal 200.

When the capturing is completed through the user terminal 200, the apparatus 100 for determining patellar dislocation receives the back image and the biometric data of the pet (S20). In FIG. 8, the back image and the biometric data are illustrated to be transmitted together. However, by first receiving the biometric data and then capturing the image, the image data may be received later or the image data may be received first.

The apparatus 100 for determining patellar dislocation recognizes a plurality of keypoints (feature points) for calculating the diagnostic angle of the patellar dislocation in the back image received through the user terminal 200 (S30). As illustrated in FIG. 4, the keypoint recognition may be performed on each of the left and right sides of the pet. The keypoint recognition may be automatically recognized by a machine learning model machine-learned based on labeled training data or a known image processing algorithm, or may also be determined by providing the guide information for selecting the keypoint to the user terminal 200 and receiving at least one of three keypoints from the user.

When some keypoints cannot be recognized in the received image, or the positions of the corresponding keypoints on both sides differ by more than the predetermined reference value, the fact that the user terminal 200 may be requested to re-capture or re-upload the image is as described above.

As described above, when the keypoints are recognized on the left and right sides, the apparatus 100 for determining patellar dislocation calculates diagnostic angles for the left and right sides, based on the recognized keypoints (S40). The diagnostic angle may be calculated as illustrated in FIG. 5.

In this case, the apparatus 100 for determining patellar dislocation may correct the diagnostic angle calculated from at least one of the left and right sides based on the height difference between the corresponding keypoints on both sides. As described above, the correction information that is the basis for correction of the diagnostic angle may be defined as a correction value or correction coefficient corresponding to the height difference value or difference range on both sides, or may be defined as the correction value or correction coefficient corresponding to the ratio (for example, d1/d2 or d2/d1) the height difference value on both sides of the plurality of keypoints.

Subsequently, the apparatus 100 for determining patellar dislocation derives the image-based patellar dislocation diagnosis results based on the calculated diagnostic angle (S50). The apparatus 100 for determining patellar dislocation may determine the progress level of patellar dislocation based on the reference angle information for each progress level of patellar dislocation.

In addition, the apparatus 100 for determining patellar dislocation evaluates the risk of joint disease based on the biometric data of the pet received from the user terminal 200 (S60). The risk of developing joint disease is evaluated based on the age, breed, BCS, clinical symptoms and past history of joint disease, lifestyle, and living environment data of the pet obtained through online questionnaires.

The apparatus 100 for determining patellar dislocation combines the image-based diagnosis result and the biometric data-based risk to finally derive the patellar dislocation diagnosis result of the pet, and transmits the final diagnostic result to the user terminal 200 (S70 and S80).

Each operation of the above-described method of diagnosing patellar dislocation in a pet may be omitted or other operations may be added as needed.

For example, the apparatus 100 for determining patellar dislocation may perform an operation of extracting an outer contour of the hind leg using an 8-way contour tracking technique from the acquired image before recognizing the keypoints in the back image, or an image pre-processing operation of using a Gaussian filter to correct an irregular contour caused by hair of a pet to smooth the contour. Alternatively, the apparatus 100 for determining patellar dislocation may recognize the keypoints in the back image and then perform the image pre-processing operation of removing the specific body area of the pet.

In addition, as described above, it further includes an operation of inputting the back image received from the user terminal 200 to a learning model trained by applying abnormal pet data and normal pet data as training data, and may be implemented to finally derive the patellar dislocation diagnosis results of the pet by combining the results of the presence or absence of patellar dislocation output from the learning model with the diagnostic angle-based diagnostic result and biometric data-based risk.

Meanwhile, the method of diagnosing patellar dislocation in a pet according to the embodiment of the present invention described above may also be implemented as a computer program stored in a computer-readable recording medium.

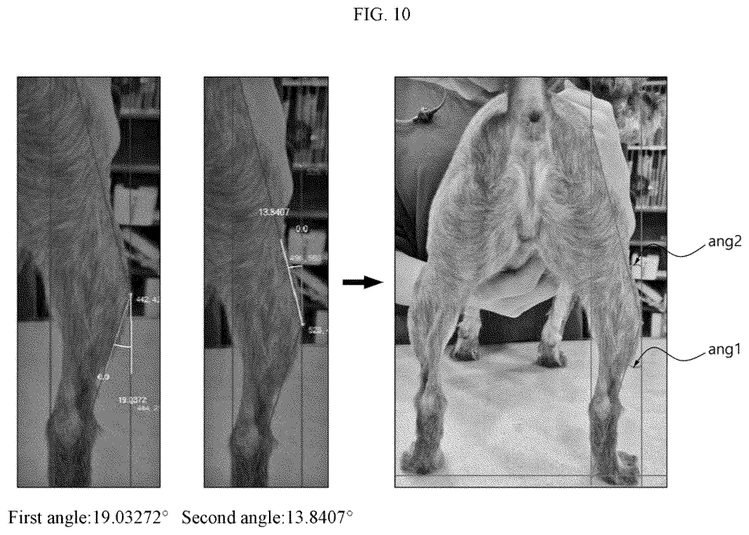

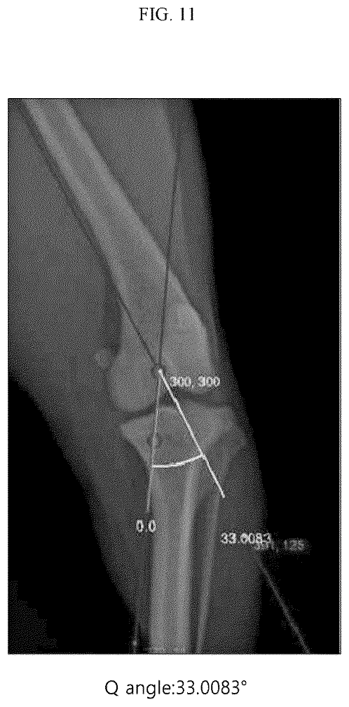

FIGS. 10 and 11 are reference diagrams for understanding the accuracy of the image-based patellar dislocation diagnosis results according to an embodiment of the present invention, FIG. 10 illustrates the diagnostic angle of patellar dislocation calculated through the back image processing according to the present invention, and FIG. 11 illustrates a quadriceps angle (QA) (hereinafter referred to as a ‘Q angle’) calculated from an X-ray image of the same pet as in FIG. 10.

For reference, the Q angle is an angle formed between a straight line connecting a rectus femoris origin and a center of a trochlear groove and a straight line connecting the center of the trochlear groove and a tibial tuberosity, and is widely used as a diagnostic indicator when diagnosing the patellar dislocation based on medical images such as X-ray, CT, and MRI. It has been proven through several papers and medical data that the Q angle is closely related to the patellar dislocation.

First, referring to FIG. 10, in the diagnostic angle of the patellar dislocation according to the present invention, it can be seen that the first angle ang1 is calculated as 19.0372°, the second angle ang2 is calculated as 13.8407°, and the diagnostic angle, which is the sum of the first angle ang1 and the second angle, is calculated as 32.8779°.

Next, referring to FIG. 11, it can be seen that the Q angle in the X-ray image was calculated to be 33.0083°, which is very similar to the diagnostic angle according to the embodiment of the present invention.

As can be seen through this, according to the apparatus and method for determining patellar dislocation in a pet according to the present invention, the presence or absence and degree of patellar dislocation may be accurately diagnosed only with digital photos using a smartphone, etc., without capturing medical images such as X-ray, computed tomography (CT), and magnetic resonance imaging (MRI).

Therefore, any user may easily determine the degree of patellar dislocation in a pet without visiting a hospital, so the apparatus and method for determining patellar dislocation in a pet according to the present invention may greatly contribute to the expansion and development of pet healthcare services.

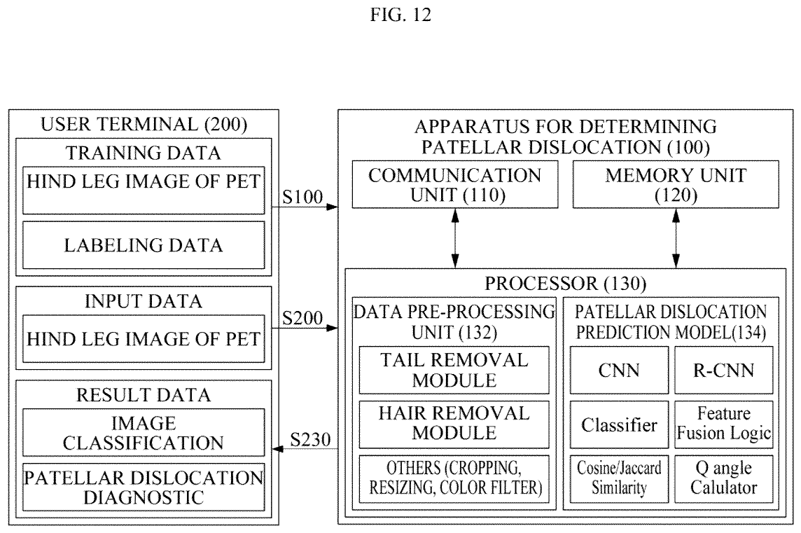

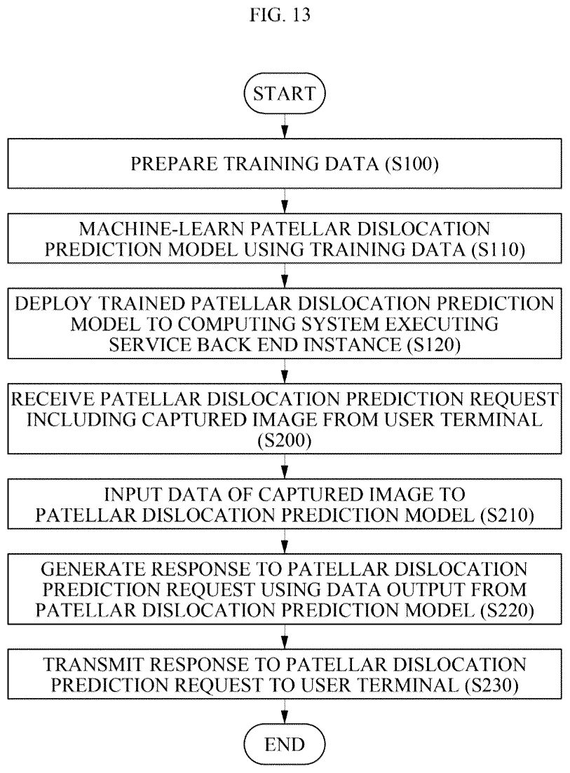

FIG. 12 is a diagram illustrating a structure of an apparatus for determining patellar dislocation in a pet using an artificial intelligence model according to another embodiment of the present disclosure, and FIG. 13 is a flowchart of a method of determining patellar dislocation in a pet according to an embodiment of the present disclosure. Hereinafter, an apparatus and method for determining patellar dislocation in a pet according to another embodiment of the present disclosure will be described with reference to FIGS. 12 and 13, respectively.

The method of determining patellar dislocation in a pet according to the present embodiment may be performed by one or more computing devices. That is, in the method of determining patellar dislocation in a pet according to the present embodiment, all operations may be performed by one computing device, or some operations may be performed by another computing device. For example, some operations may be performed by a first server system and other operations may be performed by a second server system.

In addition, as the server system is implemented on a cloud computing node, operations performed by one server system may also be performed separately on a plurality of cloud computing nodes. Hereinafter, in describing the method of determining patellar dislocation in a pet according to the present embodiment, description of the performance subject of some operations may be omitted, and in this case, the performance subject may be the apparatus 100 for determining patellar dislocation described with reference to FIG. 1.

Referring to FIGS. 11 and 13, in operation S100, training data is prepared. The training data may include a plurality of photos of a back of a pet captured from a tail to a head so that the hind legs of the pet are visible, and labeling data for each photo

The labeling data may include, for example, severity of patellar dislocation for each photo and one or more additional information. For example, the additional information may include profile information such as age and gender of a pet, and body measurement information such as height and weight. The labeling data may be, for example, composed solely of the severity of patellar dislocation for each photo.

In operation S110, a patellar dislocation prediction model using the prepared training data is machine learned. By automating the training data acquisition and establishing a metric of the training data acquisition situation, the machine learning on the patellar dislocation prediction model may be repeatedly performed periodically or aperiodically, and the timing of performing such machine learning may be determined based on the value of the metric.

Next, when the machine learning is completed, in operation S120, the trained patellar dislocation prediction model may be deployed to a computing system executing a service back-end instance of a pet care service. As described above, the computing system executing the back-end instance may be a service server.

In operation S200, when input data including a captured image of a hind leg area of a targeted pet is received from the user terminal along with the patellar dislocation prediction request, in operation S210, a back view image of a pet may be input to the patellar dislocation prediction model.

As described above, the apparatus 100 for predicting patellar dislocation includes the communication unit 110, the memory unit 120, and the processor 130. In another embodiment, the processor 130 performs an operation of executing the patellar dislocation prediction model 134 stored in the memory unit 120 to determine the patellar dislocation from input data. Since the method of operating the communication unit 110 and the memory unit 120 is the same as described above, detailed description thereof will be omitted herein.

Meanwhile, the data pre-processing unit 132 of the processor 130 may additionally perform preset pre-processing operations such as cropping, resizing, or color filter reflection on the back view image of the pet, and conversely, the data pre-processing unit 132 may perform preset pre-processing using a tail removal module and a hair removal module as processing to improve the performance of the patellar dislocation prediction model. For example, it may be pre-processing of identifying hair included in a back view image of a pet and removing a part of the hair through the hair removal module, or identifying and removing a part or all of a tail, which is a specific body area, through the tail removal module. Contents related to the pre-processing operations will be described in detail below. In operation S220, a response to the patellar dislocation prediction request may be generated using data output from the patellar dislocation prediction model, and in operation S230, a response to the patellar dislocation prediction request may be transmitted to the user terminal.

According to the present embodiment, a pet's guardian may confirm patellar dislocation-related analysis results simply by capturing a photo of a hind leg area of a pet. For example, information on the expected severity of patellar dislocation may be displayed on the user terminal.

For accurate analysis related to patellar dislocation, artificial neural network architectures unique to the present disclosure, which are robust to various appearances of a pet that may be reflected in a photo of a hind leg area of a pet and enable information reflecting anatomical characteristics of animals with patellar dislocation to be well reflected, will be described.

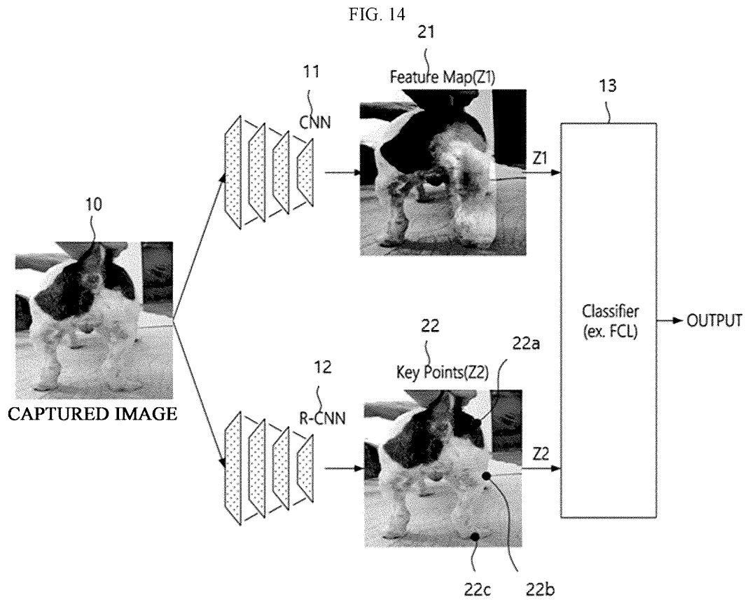

The artificial neural network architecture of the patellar dislocation prediction model executed by the processor 130, which will be described later, are elaborately designed so that the patellar dislocation prediction model may synthesize information from various perspectives to identify a skeletal structure of a hind leg area, which may be covered or concealed by long hair of a pet, to identify as accurately as possible. Hereinafter, examples of the artificial neural network architecture of the patellar dislocation prediction model that analyzes the photo of the hind leg area of the pet will be described with reference to FIGS. 14 to 23.

First, a first artificial neural network architecture of the patellar dislocation prediction model will be described with reference to FIG. 14. This artificial neural network architecture may include an image classification model that receives a captured image 10 and outputs feature vectors 21, an object detection model that receives the captured image 10 and outputs predefined keypoints 22, and a classifier 13 that performs the classification based on the feature vectors 21 and the keypoints 22. In one embodiment, the image classification model may be a CNN 11, and the object detection model may be a region with CNN (R-CNN) 12, and each artificial intelligence model is not necessarily limited thereto, but for convenience of description, the description below will be made on the assumption that the image classification model and object detection model are the CNN and the R-CNN, respectively.

The keypoint extraction method based on the R-CNN 12 may refer to various public literatures such as “https://github.com/bitsauce/Keypoint_RCNN.” In addition, the R-CNN 12 may be replaced by fastR-CNN (https://arxiv.org/abs/1504.08083), (https://arxiv.org/abs/1506.01497), etc.

In some embodiments, the machine learning process of the patellar dislocation prediction model illustrated in FIG. 14 may be a process of learning the entire CNN 11, R-CNN 12, and classifier 13 in an end-to-end manner. Since the end-to-end machine learning tends to operate well when there is a lot of labeled data on both ends of the input layer and the output layer, when a large amount of training data, for which the labeling of severity of patellar dislocation is completed, is secured, the patellar dislocation prediction model may be updated through the end-to-end machine learning.

In some other embodiments, the machine learning process of the patellar dislocation prediction model illustrated in FIG. 14 is a process of individually training each of the CNN 11, R-CNN 12, and classifier 13 as a pipeline network. In labeling each training data for machine learning, the labeling of the severity of patellar dislocation will be the most expensive task, and therefore, it will not be easy to secure the captured image for which the labeling of patellar dislocation severity is completed. The performance indicator of the overall patellar dislocation prediction may be maximized by first training the CNN 11 pipeline network and R-CNN 12 pipeline network, and then training the classifier 13 network using the training data for which the labeling of the severity of patellar dislocation is completed.

Depending on the situation in which the training data is secured, the fact that either the above-described end-to-end machine learning or machine learning in a pipeline network unit may be executed may be commonly applied to various artificial neural networks, which will be described below with reference to FIGS. 14 to 23.

The classifier 13 may be composed of various known structures, such as a fully connected layer (FCL). The classifier 13 included in the trained patellar dislocation prediction model may output probability values for each class indicating each severity level of the patellar dislocation.

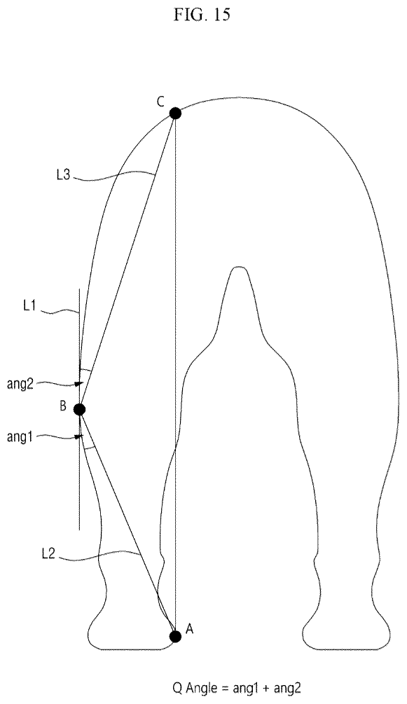

The previously defined keypoints will be exemplarily described with reference to FIG. 15. The predefined keypoints may include the first keypoint A corresponding to the innermost point of the sole of one side of the pet, the second keypoint B corresponding to the outermost point of the knee of one side of the pet, and the third keypoint C corresponding to the uppermost point among the points where the body of the pet intersects with the line perpendicular to the ground and passing through the first keypoint A.

The classifier 13 in FIG. 14 receives a feature vector 21 (Z1) output from the CNN 11 and a vector Z2 composed of a series of keypoints 22a, 22b, and 22c output from the R-CNN 12.

Meanwhile, as described above, the diagnostic angle that is an angle using the angle ang1 formed by the vertical line L1 passing through the second keypoint B and a connecting line A-B of the first keypoint and the angle ang2 formed by the vertical line L1 and a connecting line B-C of the second keypoint and the third keypoint may be input as an input feature to the classifier 13 of the patellar dislocation prediction model.

Here, the diagnostic angle may be, for example, the Q angle, which is the sum of the angles ang1 and ang2. Hereinafter, the meaning of the ‘Q angle’ described in the present disclosure will be described. The commonly used Q angle refers to an angle formed by a line connecting a center point of patella on an anterior superior iliac spine and a line connecting a center point of patella on tibial tubercle. The commonly used meaning of the Q angle refers to the web document https://www.physio-pedia.com/%27Q%27_Angle.

As illustrated in FIG. 15, the Q angle described in the present disclosure does not refer to the same angle as the commonly used Q angle. Since the commonly used Q angle is an anatomical indicator, it can only be measured on radiographs that may confirm a position of a bone. On the other hand, it has been confirmed through various cases that the Q angle described in the present disclosure is an angle that may be easily calculated through specification of R-CNN-based keypoints for the input image that is the result of general photography, and is a new angle indicator that may serve as a digital biomarker for patellar dislocation.

It should be clearly stated that the ‘Q angle’ of the present disclosure does not refer to the angle formed by the commonly used line connecting the center point of the patella on the anterior superior iliac spine and the line connecting the center point of the patella on the tibial tubercle, but is the angle of ang1+ang2 as described with reference to FIG. 5.

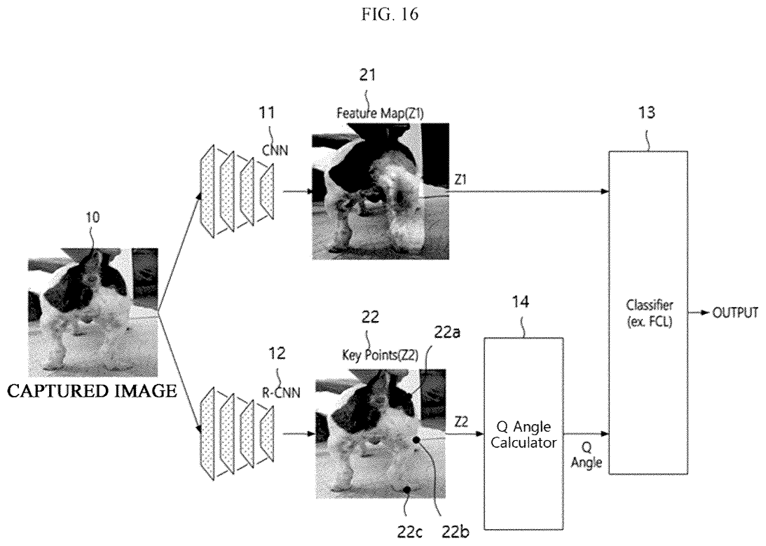

FIG. 16 illustrates a second artificial neural network architecture of the patellar dislocation prediction model including the output feature vector 21 (Z1) of the CNN and the classifier 13 receiving the Q angle. As illustrated in FIG. 16, a Q angle calculation logic 14 that receives the keypoints 22 output from the R-CNN 12 and calculates the Q angle in the above-described manner may be embedded in the second artificial neural network architecture illustrated in FIG. 16.

The classifier 13 illustrated in FIG. 16 receives the Q angle itself, which has a high causal relationship with the severity of patellar dislocation, as one of the input features, so it may show a more accurate patellar dislocation severity classification performance compared to the classifier that receives the keypoints illustrated in FIG. 14.

When the hair of the pet is short or thin and the skeletal structure of the pet is relatively visible in the photo, a series of keypoints 22a, 22b, and 22c output from the R-CNN 12 or the Q angle obtained from the keypoints 22a, 22b, and 22c will be relatively accurate.

On the other hand, when the hair of the pet is long or the pet is fat and the skeletal structure of the pet is not relatively visible in the photo, a series of keypoints 22a, 22b, and 22c output from the R-CNN 12 or the Q angle obtained from the keypoints 22a, 22b, and 22c will be inaccurate.

Therefore, in preparation for this case, the above-described first artificial neural network architecture and second artificial neural network architecture includes the classifier 13 that receives the feature vector 21 (Z1) extracted from the input captured image 10 through the CNN 11 along with the series of keypoints 22 (Z2) output from the R-CNN 12 or the Q angle obtained from the keypoints 22

Even when the skeletal structure is not relatively visible in the photo, in order for the feature vector 21 (Z1) extracted from the input captured image 10 through CNN 11 to well express patellar dislocation-related feature information, in some embodiments, the patellar dislocation prediction model trained through the end-to-end machine learning may be used. That is, in some embodiments, when a hair length or obesity level is preliminarily calculated for the captured image 10 captured by the user terminal and the hair length value is shorter than the reference value and the obesity level is less than the reference value, the patellar dislocation prediction model generated in a manner in which the pipeline networks are individually machine-learned may be used, and when the hair length is longer than the reference value or the obesity level is higher than the reference value, the patellar dislocation prediction model trained through the end-to-end machine learning may be used.

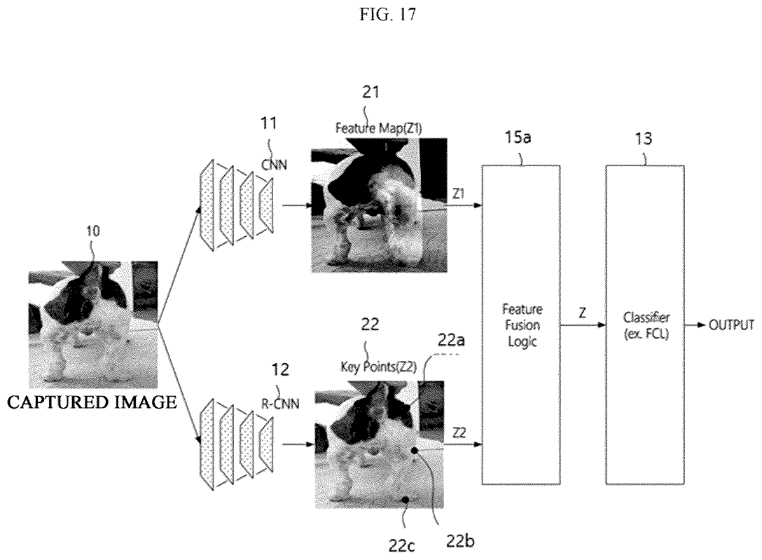

Hereinafter, third to ninth artificial neural network architectures will be described with reference to FIGS. 17 to 23. Since the vector of the keypoints or the vector of the Q angle and the CNN output feature vector that are targets of feature fusion are all information that may affect the patellar dislocation severity classification, the third to ninth artificial neural network architectures are the result of the feature fusion, and may show more accurate patellar dislocation severity classification by performing the classification using the output single feature vector Z.

The feature fusion may be performed, for example, by connecting each input feature using a concatenation method, but the feature fusion method may be different according to the structure of each of the artificial neural network architectures. Hereinafter, while describing each artificial neural network architecture, specific feature fusion application methods will be described.

According to the third artificial neural network architecture illustrated in FIG. 17, the vector Z2 of the keypoints and the CNN output feature vector Z1 are input to a feature fusion logic 15a, a single vector Z is output from the feature fusion logic 15a, and the single vector Z is input to the classifier 13.

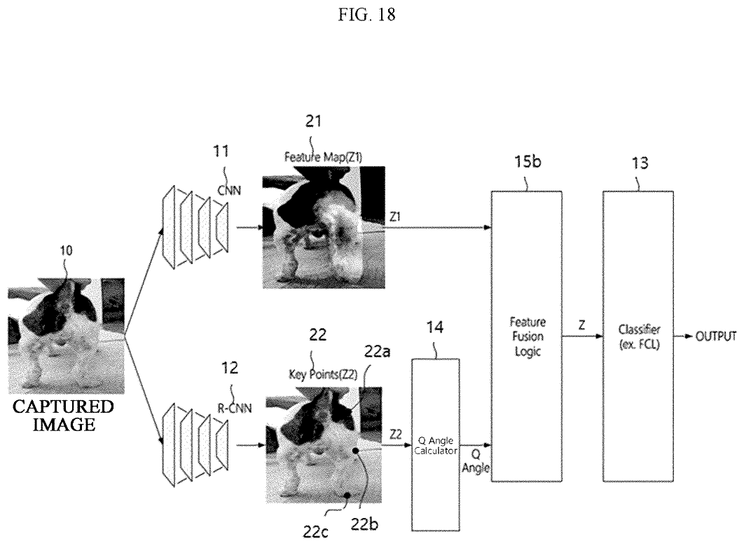

According to the fourth artificial neural network architecture illustrated in FIG. 18, a vector representing the Q angle calculated by a Q angle calculation logic 14 and the CNN output feature vector Z1 are input to a feature fusion logic 15b, and the single vector Z is output from the feature fusion logic 15b, and the single vector Z is input to the classifier 13.

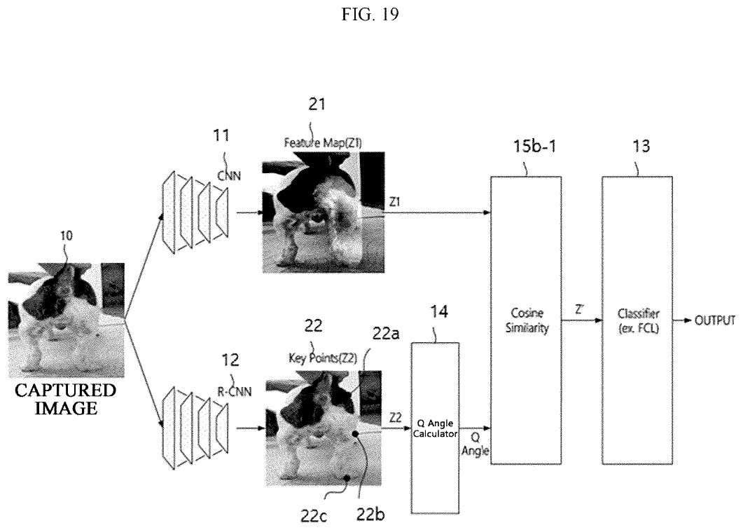

The feature fusion logic 15b may output a similarity between two fusion target features in a feature space. For example, like the fifth artificial neural network architecture illustrated in FIG. 19, a feature fusion logic 15b-1 may output a cosine similarity Z′ of the input feature vectors.

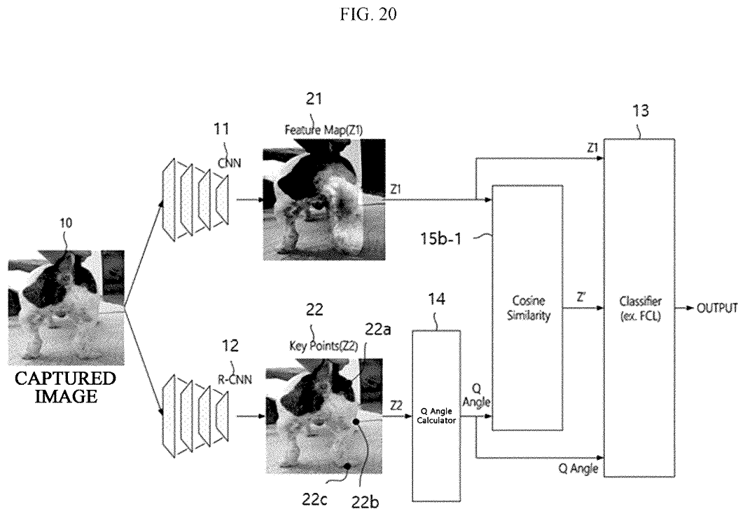

In some embodiments, to perform the classification by considering more information than the cosine similarity Z′ output from the feature fusion logic 15b-1, like the sixth artificial neural network architecture illustrated in FIG. 20, the classifier 13 may additionally receive a vector representing the Q angle and a CNN output feature vector Z1 as the input feature. That is, the classifier 13 included in the sixth artificial neural network architecture may receive the vector representing the Q angle, the CNN output feature vector Z1, and the cosine similarity Z′ as the input feature.

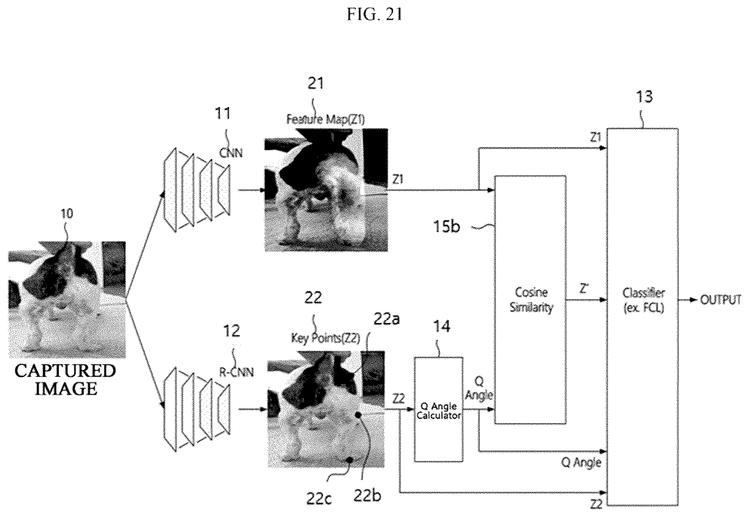

The classifier 13 of the seventh artificial neural network architecture illustrated in FIG. 21 may receive a total of four input features, including the vector representing the keypoints, the vector representing the Q angle, the CNN output feature vector Z1, and the cosine similarity Z′.

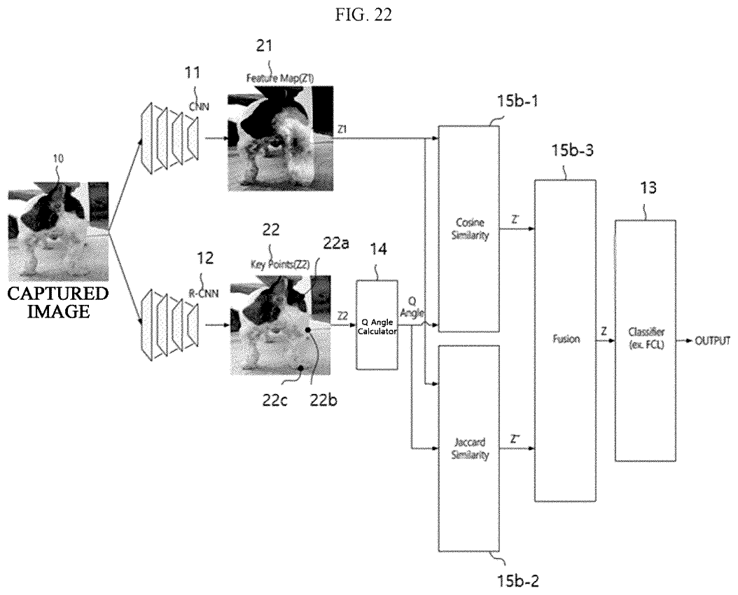

Meanwhile, as the feature fusion logic 15b, in addition to the cosine similarity, Jaccard similarity or Pearson correlation coefficient may be output. Taking this into account, the eighth artificial neural network architecture illustrated in FIG. 22 outputs the cosine similarity Z′ as a result of feature fusion of the CNN output feature vector Z1 and the vector of the Q angle, outputs the Jacquard similarity Z″ as a result of feature fusion of the CNN output feature vector Z1 and the vector of the Q angle, performs the feature fusion 15b-3 of the cosine similarity Z′ and the Jacquard similarity Z″ once again, and as a result, outputs the single vector Z, and inputs the single vector Z to the classifier 13.

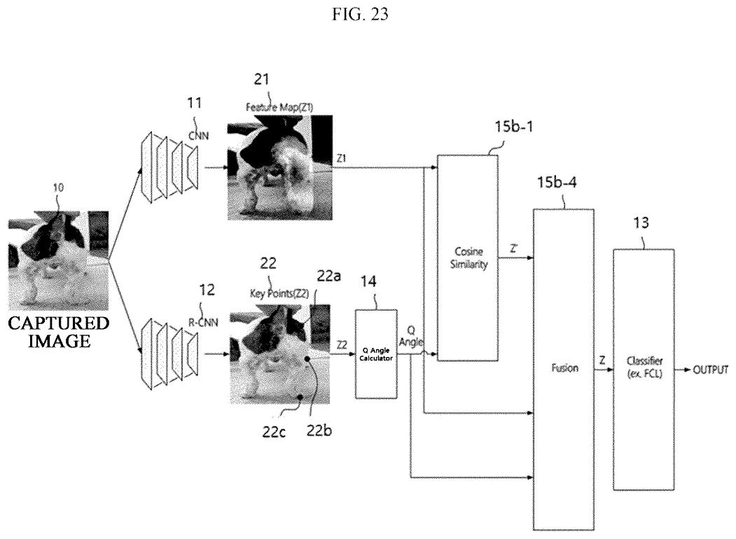

In the ninth artificial neural network architecture illustrated in FIG. 23, a feature fusion logic 15b-4 may receive cosine similarity Z′, a vector representing the Q angle, and the CNN output feature vector Z1. The feature fusion logic 15b-4 may perform the feature fusion by assigning different weights to each of the cosine similarity Z′, the vector representing the Q angle, and the CNN output feature vector Z1. In this case, by considering that information damage may occur during the calculation of the cosine similarity Z′, the weight assigned to the cosine similarity Z′ may be lower than the weight assigned to the vector representing the Q angle and the weight assigned to the CNN output feature vector Z1.

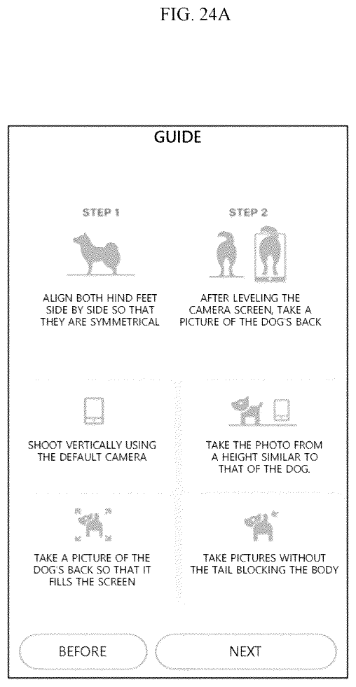

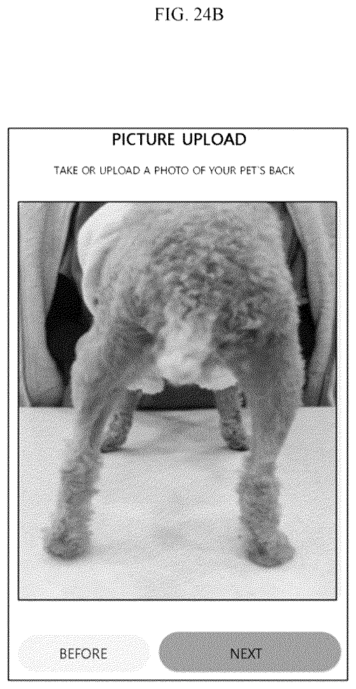

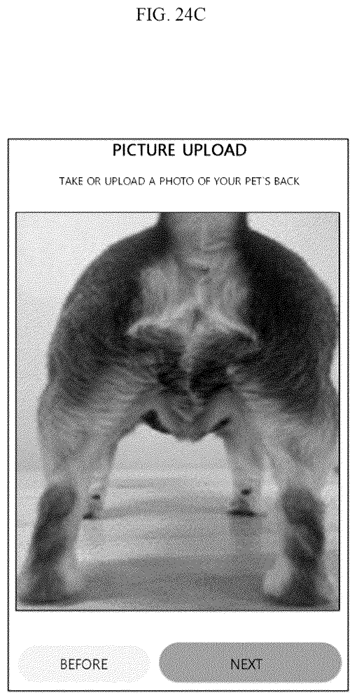

FIGS. 24A to 24C to 27 are diagrams illustrating the user interface screen displayed on the user terminal according to the input of the back view images of different pets in an embodiment of the present disclosure.

As described above, the apparatus 100 for determining patellar dislocation acquires the back view image including the hind leg of the pet area from the user terminal 200. The back view image of the pet may be a photo captured directly through the camera module provided in the user terminal 200, pre-stored in the internal memory of the user terminal 200, or an image photo transmitted from an external device. In the following embodiment, the user interface screen will be described based on the back view image of the pet generated by capturing from the user terminal 200.

Referring to FIGS. 24A to 24C, the user may capture the hind leg area according to the capturing guide to generate the back view image of the pet. When the area required to determine the patellar dislocation is not included in the back view image, the accuracy of the result of determining the patellar dislocation is significantly reduced, so the capturing guide may be provided to a user by including multiple steps to facilitate acquiring the optimal back view image.

For example, as illustrated in FIG. 24A, it may include aligning both hind legs so that the hind feet of the pet are symmetrical when capturing a photo (step 1), adjusting the camera screen to the height and level where the pet is located and then capturing the back view of the pet (step 2), and the like. Additionally, by using the default camera, it is possible to guide the camera settings so that the pet fills the screen in a vertical screen, or guide precautions not to cover the hind legs with the tail.

The apparatus 100 for determining patellar dislocation of the present disclosure may provide detailed capturing guide information and precautions information to the user terminal 200 before capturing the back view image, thereby lowering the probability of needing to re-capture a photo and improve the accuracy of determining patellar dislocation.

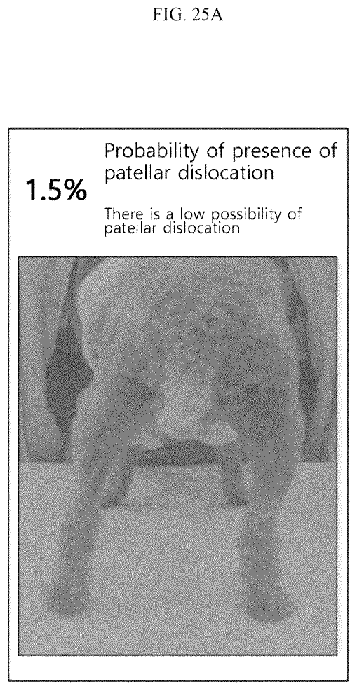

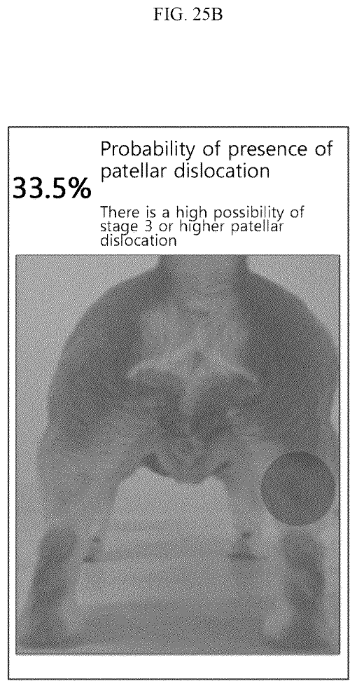

The acquired back view image of the pet is input to the trained patellar dislocation prediction model, and the apparatus 100 for determining patellar dislocation outputs the patellar dislocation information of the pet corresponding to the back view image. The dislocation information is the patellar dislocation-related information, and as illustrated in FIGS. 25A and 25B, may include at least one of the probability of presence of patellar dislocation in a pet, the degree of progress of patellar dislocation, and the patellar dislocation location visualization image. As various types of visualization information is included in the patellar dislocation information, users may intuitively check whether the patellar dislocation has occurred, the position where the patellar dislocation has occurred, and the degree of progress of patellar dislocation, etc., helping them take immediate action for the pet.

In addition, the additional information described below is provided to the user terminal 200 together with the output patellar dislocation information, and the additional information of the currently targeted pet based on the patellar dislocation information may include the patella health score information, the recommended test guide information, the lifestyle guide information, the customized product recommendation information, the expert consultation information, etc.

Meanwhile, the input back view image is the back view image including the diagnostic angle related to the patella of the pet. The fact that the diagnostic angle included in the back view image may be different depending on the pet to be captured and the diagnostic angle includes the Q angle calculated by summing the angles formed by the keypoint connection line is as described above.

For example, when the back view image is generated by capturing the hind leg area for each of the captured targeted pets A and B, the first back view image of the pet A may represent the first diagnostic angle with respect to the patella, and the second back view image of pet B may represent the second diagnostic angle that is different from the first diagnostic angle with respect to the patella.

As the plurality of back view images representing different diagnostic angles are each input to the patellar dislocation prediction model, the apparatus 100 for determining patellar dislocation may output different patellar dislocation information. That is, the apparatus 100 for determining patellar dislocation may output the first dislocation information for the first back view image representing the first diagnostic angle, and output the second dislocation information for the second back view image representing the second diagnostic angle.

Referring back to FIGS. 24A to 24C, FIG. 24B illustrates the first back view image of the pet A including the first diagnostic angle information, and FIG. 24C illustrates the user terminal screen representing the second back view image of the pet B including the second diagnostic angle information.

In this case, each piece of diagnostic angle information is not the diagnostic angle visually displayed on the back view image, but may be calculated by the user or included in the form of numerical information on the diagnostic angle obtained in advance by prior determination by the patellar dislocation prediction model.

As the first diagnostic angle information and the second diagnostic angle information represent different diagnostic angles, the first dislocation information (see FIG. 25A) and the second dislocation information (see FIG. 25B) show different results, as illustrated in FIGS. 25A and 25B. Specifically, in the first dislocation information (see FIG. 25A), the probability of presence of dislocation is only 1.5%, while, in the second dislocation information (see FIG. 25B), the probability of presence of dislocation is 33.5%, and it is confirmed that the dislocation occurs in the right hind leg along with the degree of progress of stage 3 or higher, giving different results.

Meanwhile, it is difficult to conclude that the result of outputting different types of patellar dislocation information in two back view images of a pet including different types of diagnostic angle information is not necessarily caused by the diagnostic angle information. Therefore, it is necessary to consider the remaining variable factors excluding the diagnostic angle information included in the back view image.

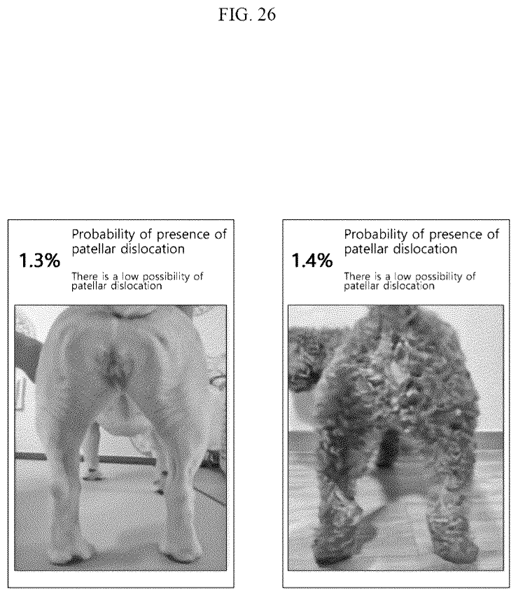

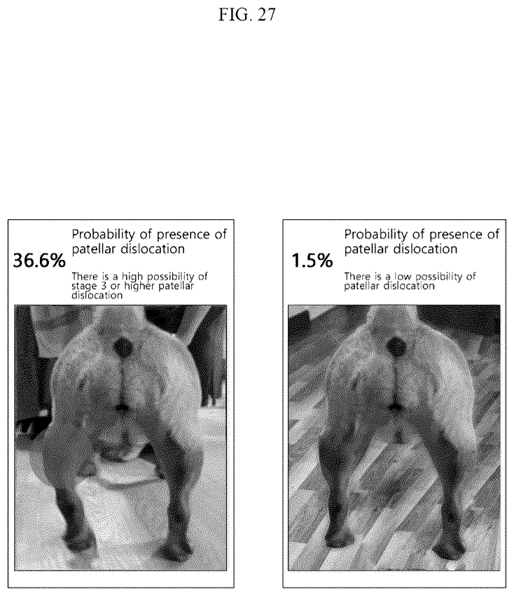

FIG. 26 is a diagram illustrating patellar dislocation information on each back view image of different breeds of pets having the same diagnostic angle information in the embodiment of the present disclosure, and FIG. 27 is a diagram illustrating patellar dislocation information on each back view image of the same breed of pets having different diagnostic angle information in another embodiment of the present disclosure.

Referring to FIG. 26, each targeted pet captured in the same environment is a different breed, and includes the same diagnostic angle information in the captured back view image. On the other hand, FIG. 27 is the back view image of the pet of the same breed acquired in the same environment and includes different diagnostic angle information.

As a result, the result of determining the patellar dislocation within the corresponding range of 1.3% and 1.4% is derived for the back view images of the pets of different breeds including the same diagnostic angle information, respectively, and the result of determining different patellar dislocation is derived at 36.6% and 1.5%, respectively, for the back view image of the pet of the same breed including different diagnostic angle information. This means that, compared to variable factors such as the breed or environment, the diagnostic angle information is a key element with high weight or relevance in deriving the patella determination result.

Therefore, by using the apparatus for determining patellar dislocation of the present disclosure, highly accurate patellar dislocation information is output, and regardless of the variation of other fluctuation elements, by using the back view image including different diagnostic angle information of the pet, it may be easily confirmed whether the apparatus for determining patellar dislocation of the present disclosure has been clearly implemented.

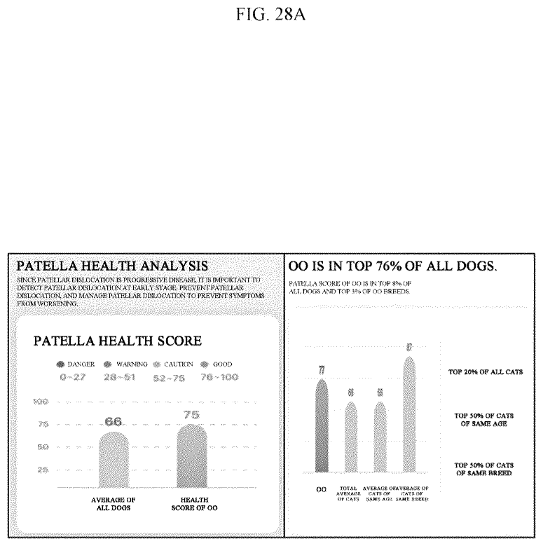

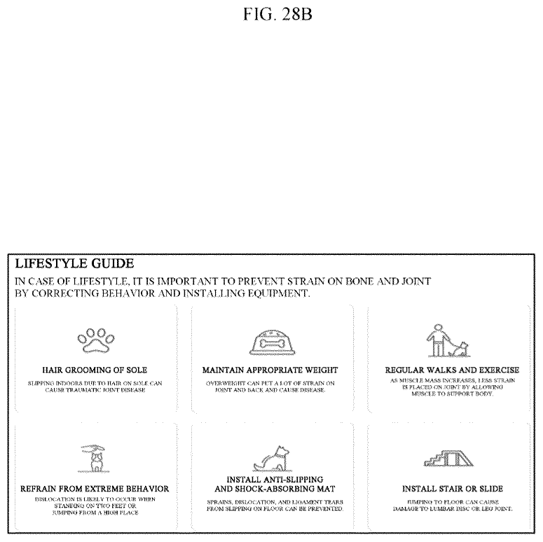

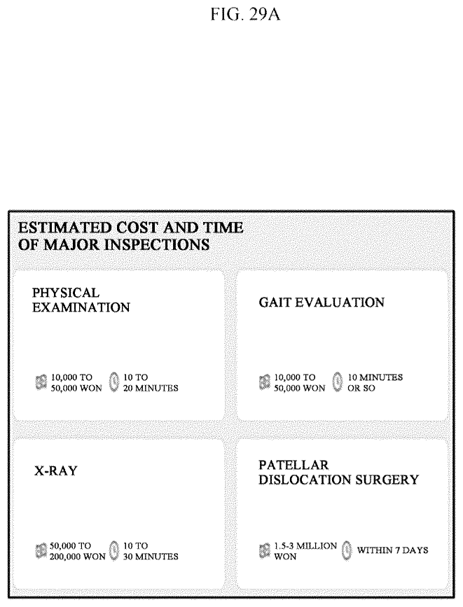

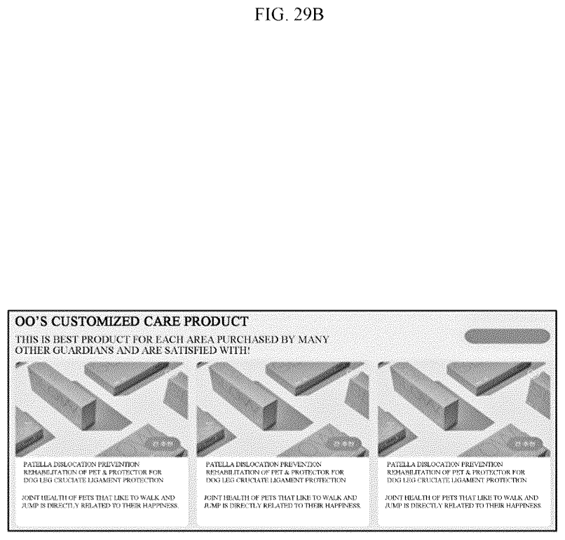

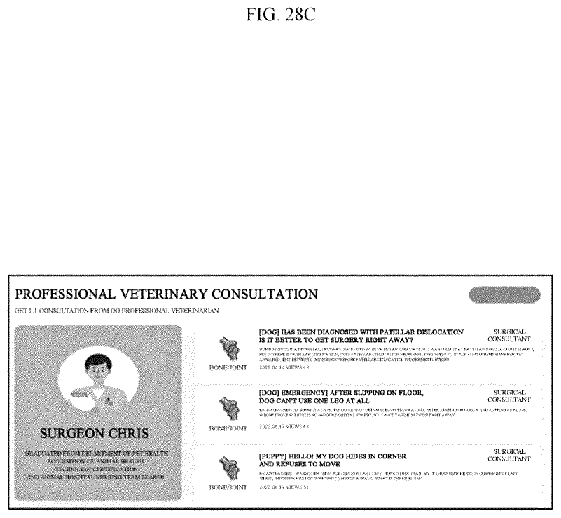

FIGS. 28A and 28B and FIGS. 29A to 29C are diagrams illustrating additional information screens provided to the user terminal according to the dislocation information in an embodiment of the present disclosure.

Referring to the drawings, the additional information may include patella health score information, lifestyle guide information, recommended test guide information, customized product recommendation information, expert consultation information, etc., of the back view image of the targeted pet based on the dislocation information.

Specifically, the health score information (see FIG. 28A) provides the quantified health score and grade of the targeted pet according to the dislocation information, and the average health score of all dogs and percentile information of the targeted pet may also be provided together to facilitate comparison.

The lifestyle guide information (see FIG. 28B) includes guide information on the lifestyle habits that the targeted pet should pay attention to on a daily basis to prevent the patellar dislocation. For example, the lifestyle guide information includes hair grooming of a sole of a pet, weight maintenance, and regular exercise, etc.

The recommended test guide information (see FIG. 29A) includes offline patellar dislocation-related test types, test costs, and test time information through hospital visits according to dislocation information, and includes, for example, physical examination (10,000 to 50,000 won, 10 to 20 minutes), gait evaluation (10,000 to 50,000 won, approximately 10 minutes), X-ray (50,000 to 200,000 won, 10 to 30 minutes), and patellar dislocation surgery (1.5 to 3 million won, within 7 days), etc.

The customized product recommendation information (see FIG. 29B) is information that recommends patella care-related auxiliary products according to the dislocation information. Only the auxiliary product-related information may be provided, and a sales link may be provided directly so that it can be easily forwarded to a shopping mall that sells auxiliary products.

The expert consultation information (see FIG. 29C) provides professional veterinary consultation services based on the dislocation information, and may include some personal information of professional veterinarians in consideration of the ease of selection by the pet's guardians.

In this way, by providing additional information to the user terminal 200 along with the dislocation information, various types of information may be provided to the pet's guardians with low knowledge of joint diseases and inexperienced coping skills, and at the same time, help for rapid response.

The service operator of the apparatus for determining patellar dislocation may also provide direct shopping services or consultation services in relation to customized product recommendation information and expert consultation information, or utilize the direct shopping services or consultation services for business through partnerships with other companies.

In the above, the additional information is limited and listed, but is not necessarily limited thereto, and the additional information provided may include information on all services that may be provided using the dislocation information.

Meanwhile, when the hind leg area included in the back view image of the pet is covered by the specific body area or a problem occurs due to an abnormal capturing angle, the accuracy of the patellar dislocation information is significantly reduced.

In order to solve this problem and improve the performance of the patellar dislocation prediction model, the capturing guide is presented to horizontally calibrate the photo capturing angle in the operation of acquiring the back view image, or the method including the pre-processing process is proposed. Hereinafter, the method of operating a pre-processing process will be described in detail.

As described above, the apparatus 100 for determining patellar dislocation of the present disclosure may additionally perform the preset pre-processing operation such as cropping, resizing, or color filtering reflection on the back view image of the pet through the data preprocessing unit 132 of the processor 130.

In one embodiment, the pre-processing operation described above may be a pre-processing operation that detects a pet in the back view image of the pet to remove background noise (for example, people, objects, etc.) included in the back view image, and then excludes the background as much as possible in the process of resizing the back view image and secures the resolution of pixels included in the detection area of the cropped pet above a preset reference value. In another embodiment, the data pre-processing unit 132 may perform the pre-processing using the tail removal module and the hair removal module before the patellar dislocation prediction model outputs the dislocation information. For example, it may be pre-processing of removing a part of hair identified in the back view image of the pet through the hair removal module, or identifying and removing a part or all of the tail, which is the specific body area, through the tail removal module.

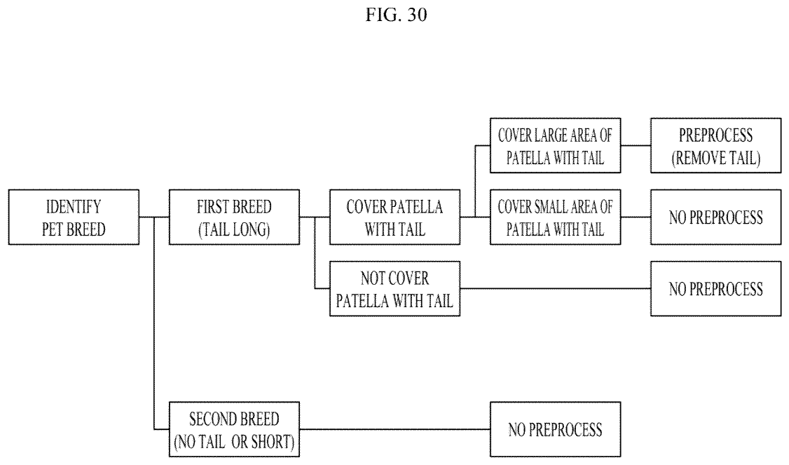

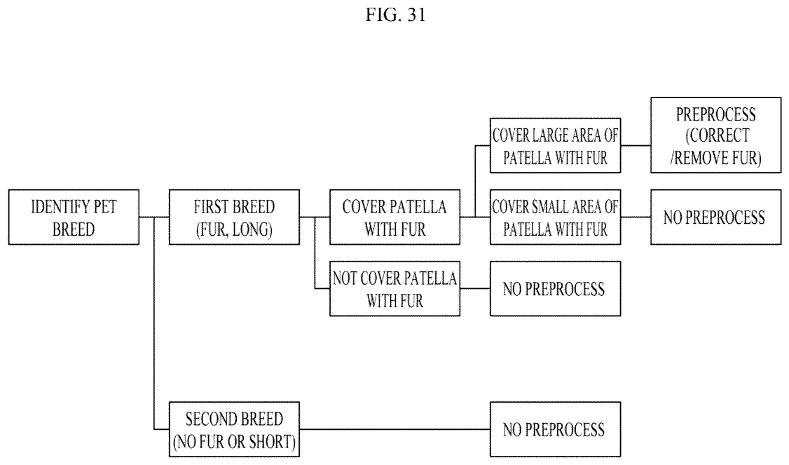

FIG. 30 is a flowchart illustrating a pre-processing method that is a method using a tail removal module in an embodiment of the present disclosure, and FIG. 31 is a flowchart illustrating a pre-processing method that is a method using a hair removal module in an embodiment of the present disclosure.

When the pre-processing is performed on all the back view images of the pet, the excessive load is applied to the apparatus 100 for determining patellar dislocation, which may reduce the processing performance. Accordingly, it is possible to improve the efficiency of the apparatus 100 for determining patellar dislocation when the pre-processing is performed only on the back view image that satisfies specific conditions.

Referring to FIG. 30, the apparatus 100 for determining patellar dislocation identifies the breed of the targeted pet. The breed of the targeted pet may be identified based on the subscription information of the pet obtained at the subscription stage, or may be identified using the known object classification artificial intelligence model, but various object identification methods not described may be used.

The data pre-processing unit 132 of the apparatus 100 for determining patellar dislocation further includes the operation of performing pre-processing based on the identified breed of the pet. For example, when the pet included in the back view image is a first breed with a long tail, the specific body area of the pet is removed depending on whether the hind leg area is covered in the back view image, and when the pet included in the back view image is a second breed with no tail or a short tail, the specific body area is not removed from the back view image.

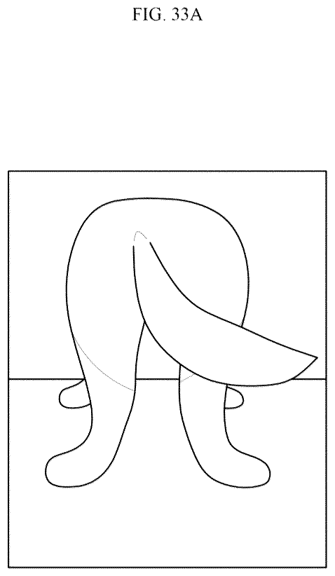

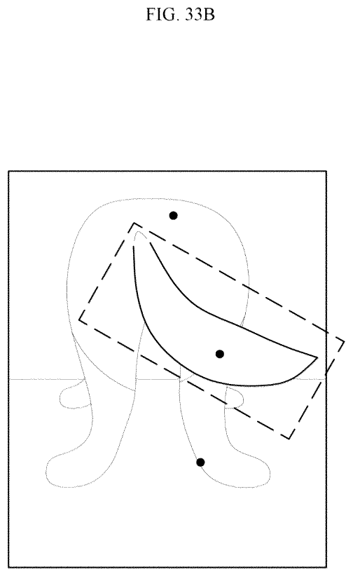

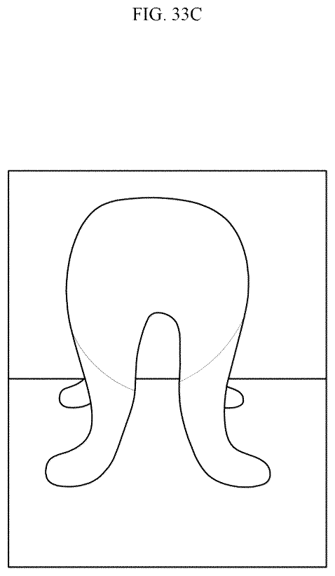

Here, the specific body area of the pet may be the tail area, and the method of determining whether the hind leg area is covered by the tail will be described below in FIGS. 33A to 33C.

Next, when the apparatus 100 for determining patellar dislocation determines that the hind leg area is covered by the tail in the back view image, it may determine whether to perform the preprocessing based on the covered degree.

In detail, in the embodiment, the tail removal module of the data pre-processing unit 132 may determine the part where the hind leg area overlaps with the tail area, and when the overlapping part exceeds the preset standard value, the covered degree is determined to be large, so the pre-processing of removing part or all of the tail area from the back view image may be performed, and when the overlapping part is less than or equal to the reference value, the pre-processing may not be performed.

The hair removal module of the data pre-processing unit 132 also performs the pre-processing based on the breed of the identified targeted pet, similar to the tail removal module. Referring to FIG. 31, when the pet included in the back view image is the first breed with long hair, the specific body area of the pet is removed depending on whether the hind leg area is covered in the back view image, and when the pet included in the back view image is the second breed with no or short hair, the specific body area is not removed from the back view image.

Here, the specific body area of the pet may be a hair area, and when it is determined that the hind leg area is covered by hair in the back view image, the apparatus 100 for determining patellar dislocation may determine whether to perform the pre-processing based on the amount of covered hair.

In detail, the hair removal module of the data pre-processing unit 132 determines the part where the specific area of the hind leg area overlaps the hair area, and if the overlapping part exceeds the preset standard value, the covered degree is determined to be large, so the pre-processing of correcting the hair length or removing the hair is performed, and when the overlapping part is less than or equal to the reference value, the pre-processing may not be performed.

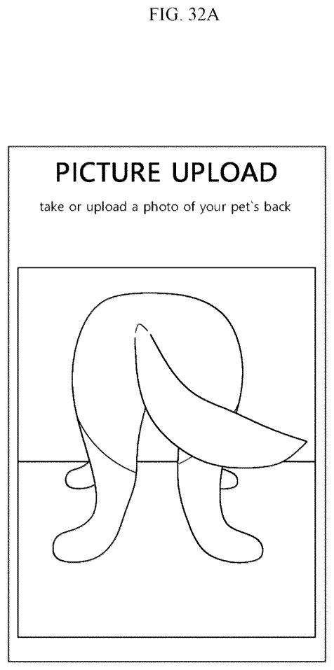

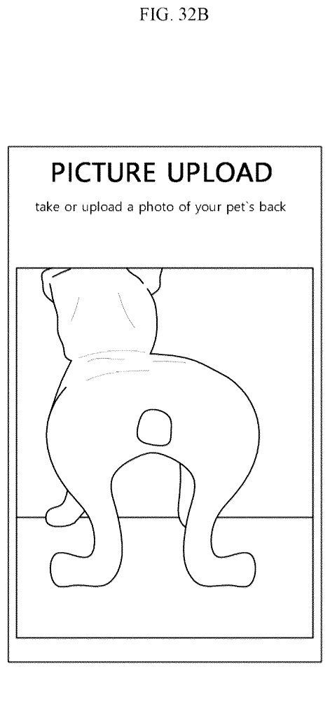

FIGS. 32A and 32B to FIGS. 34A and 34B are diagrams illustrating the user interface screen of the user terminal displaying the pre-processing process of the apparatus for determining patellar dislocation according to an embodiment of the present disclosure.

FIGS. 32A and 32B to FIGS. 34A and 34B illustrate the pre-processing method that is a method using the tail removal module or hair removal module among various embodiments. Referring to FIGS. 32A and 32B, FIG. 32A is a user terminal screen showing a back view image of a pet of a first breed with a long tail, and FIG. 32B is a user terminal screen showing a back view image of a pet of a second breed with a short tail.

According to the method of operating a tail removal module described above, the pre-processing may not be performed on the back view image of FIG. 32B which is the second breed, and it may be determined whether the pre-processing is only performed on the back view image of FIG. 32A which is the first breed.

Looking at whether the pre-processing is performed on the first breed (see FIG. 32A) illustrated in FIGS. 33A to 33C, the hind leg area is covered by the tail area in the back view image (see FIG. 33B), so the pre-processing operation of removing (see FIG. 33C) the specific body area of the pet is performed.

Hereinafter, the method of determining whether the hind leg area is covered by the tail area will be described.

The apparatus 100 for determining patellar dislocation extracts the estimated keypoint related to the knee joint from the back view image using the object detection model of the patellar dislocation prediction model. It is revealed that the estimated keypoint is a plurality of feature points extracted from the back view image to determine whether to perform the pre-processing, and corresponds to the keypoint for calculating the diagnostic angle used in the present disclosure, but the keypoint is a feature point extracted after the pre-processing is performed on the back view image, and the estimated keypoint is a feature point extracted for pre-processing before performing the pre-processing and is composed of different concepts. Meanwhile, unlike the keypoint, the estimated keypoint may be generated at inaccurate positions as the specific body area of the pet is covered.

Thereafter, the data pre-processing unit 132 extracts the tail area from the back view image of the pet. In one embodiment, the data pre-processing unit 132 may further include the image classification model, and the image classification model may include various computer vision technologies that may extract the region of interest within the image, such as image classification, object detection, semantic segmentation, and instance segmentation. When the image classification model is the object detection model, the data pre-processing unit 132 may classify the image by generating a bounding box for the tail area included in the back view image.

The operation of performing the pre-processing by the data pre-processing unit 132 compares the position of at least one of the plurality of estimated keypoints with the position of the tail area, and determines that the hind leg area is covered when the position of the tail area corresponds to the position of at least one of the estimated keypoints.

Thereafter, the data pre-processing unit 132 may determine that the covered degree is high when the number of estimated keypoints corresponding to the position of the tail area among the plurality of estimated keypoints or an area overlapping with an area of a certain range formed by the estimated keypoints is greater than or equal to a predetermined reference value, and perform the pre-processing.

In this way, when it is determined that the specific body area of the pet covers the hind leg area, the data pre-processing unit 132 completes the pre-processing by removing a part or all of the specific application area.

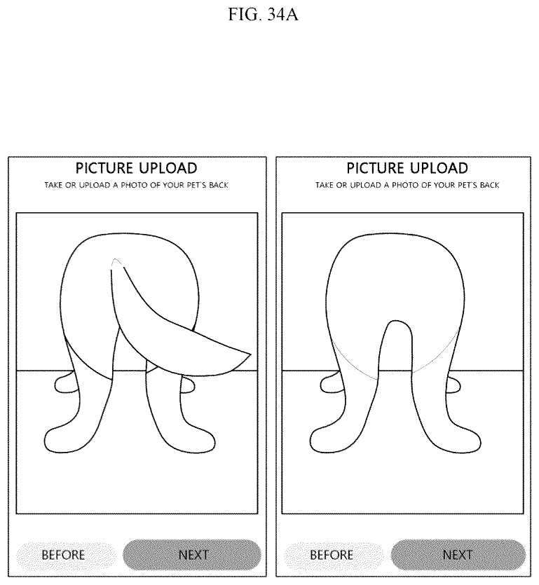

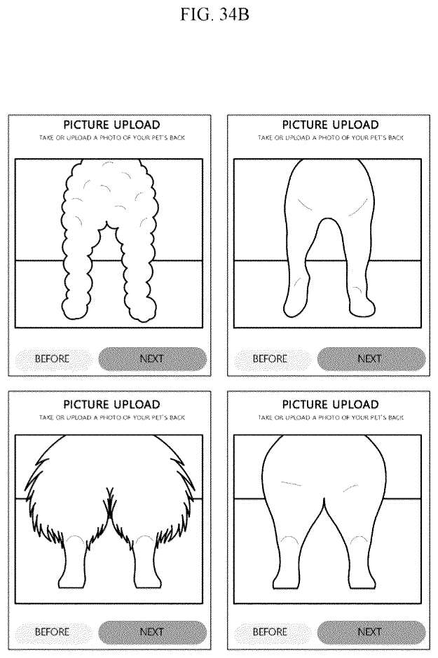

FIGS. 34A and 34B are diagrams illustrating the back view images of the pet before and after the pre-processing in an embodiment of the present disclosure.

Referring to the drawings, FIG. 34A is a diagram illustrating the pre-processing results using the tail removal module, and FIG. 34B is a diagram illustrating the pre-processing results using the hair removal module.

The method of determining whether the hind leg area is covered by the hair area may compare the position of the extracted estimated keypoint with the position of the hair area to determine whether the hind leg area is covered, as in the method of determining a tail area.

In an embodiment, the hair area of the pet may extract an outer contour of a hind leg using a LadderNet model or an 8-way contour tracking technique, and by changing the length of the hair using Gaussian blurring, averaging filter, etc., as the method of removing a hair area, it is possible to correct the outline of the body of the pet to be flat and smooth.

In this way, by making the position of the patella clearly visible through the pre-processing, it is possible to more accurately acquire the position of the keypoint position and improve the accuracy of the diagnostic angle and dislocation information.

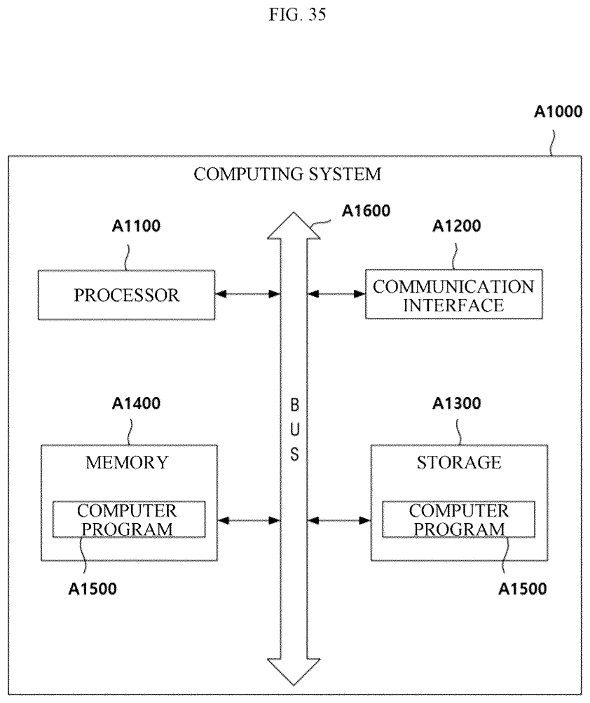

FIG. 35 is a hardware configuration diagram of a computing system according to some embodiments of the present disclosure. A computing system A1000 in FIG. 35 includes one or more processors A1100, a system bus A1600, a communication interface A1200, a memory 1400 into which the computer program A1500 executed by the processor 1100 is loaded, and a storage 1300 that stores the computer program A1500. For example, the computing system in FIG. 35 may be the apparatus for determining patellar dislocation described with reference to FIG. 1.

The processor 1100 controls an overall operation of each component of the computing system 1000. The processor 1100 may perform operations on at least one application or program to execute methods/operations according to various embodiments of the present disclosure.

The memory A1400 stores various types of data, commands and/or information. The memory A1400 may load one or more computer programs A1500 from the storage 1300 to execute methods/operations according to various embodiments of the present invention. In addition, the memory A1400 may load definition data of the patellar dislocation prediction model described with reference to FIGS. 3 to 13 and a runtime for executing the patellar dislocation prediction model.

The definition data of the patellar dislocation prediction model may be data that expresses the artificial neural network architecture that includes a CNN that outputs a first feature vector, R-CNN that outputs predefined keypoints, and a classifier that performs classification based on the first feature vector and the keypoint.

In addition, the definition data of the patellar dislocation prediction model may be one including the artificial neural network architecture that includes a classifier that performs the classification using the feature vector of the input image output from the CNN and the Q angle corresponding to the hind leg image of the pet of the input image. In this case, the Q angle may be an angle calculated using the first keypoint corresponding to the innermost point of the sole of one side of the pet, the second keypoint corresponding to the outermost point of the knee of one side of the pet, and the third keypoint corresponding to the uppermost point among the points where the body of the pet intersects with the line perpendicular to the ground and passing through the first keypoint.

The storage 1300 may non-transitorily store one or more computer programs A1500. The computer program A1500 may include one or more instructions implementing methods/operations according to various embodiments of the present disclosure. When the computer program A1500 is loaded into the memory A1400, the processor 1100 may perform the methods/operations according to various embodiments of the present disclosure by executing the one or more instructions.

For example, the computer program A1500 may include instructions to acquire the captured image of the hind leg of the pet, and instructions to input data from the acquired captured image of the hind leg to the patellar dislocation prediction model previously generated through the machine learning and generate the patellar dislocation-related analysis results using data output from the dislocation prediction model.

In some embodiments, the computing system 1000 described with reference to FIG. 35 may be configured using one or more physical servers included in a server farm based on cloud technology such as a virtual machine. In this case, at least some of the processor 1100, the memory A1400, and the storage A1300 among the components illustrated in FIG. 14 may be virtual hardware, and the communication interface A1200 may also be composed of virtualized networking elements such as a virtual switch.

Although operations are illustrated in the drawings in a specific order, it should be understood that the operations do not need to be performed in the specific order illustrated or sequential order or that all illustrated operations should be performed to obtain the desired results. In specific situations, multitasking and parallel processing may be advantageous.
[2]

Meanwhile, even for pets, obesity may be the root cause of diseases such as arthritis, diabetes, urinary disease, disc disease, chronic renal failure, hypothyroidism, cardiovascular abnormalities, asthma, liver disease, gallbladder dysfunction, and cancer. There are approximate reference weights determined based on breed, genders, ages, etc., of pets, but even for pets of the same species and gender, there are significant differences in body shape between individuals. In particular, there is no clear method of measuring height and appropriate weight of a pet as clearly as a human. Therefore, the method of measuring obesity in pets based solely on body weight is not effective.

Currently, obesity in pets is measured by an expert directly touching a body and measuring body circumference. The obesity measurement using the body circumference of the pet is implemented in a way to measure circumferences of several points on the body and compare ratios of the circumferences.

Meanwhile, diagnostic assistance technologies based on artificial intelligence technology are provided. A representative example is machine learning-based artificial intelligence that predicts the likelihood of developing a specific disease or its severity by analyzing photos of problem areas. In situations where it is difficult to visit a veterinary hospital, artificial intelligence is provided to predict the likelihood of developing a specific disease or its severity by analyzing images of targeted pets taken by a pet's guardian using a smartphone, etc.

However, basic and elemental technologies for photographing pets' bodies with a terminal and analyzing obesity in pets using artificial intelligence did not exist in Korea, and there was little discussion on the use of such artificial intelligence regarding the subject thereof.

Pets referred to in the present disclosure may include various species of pets whose obesity level may be determined not only by height and weight but also by a body shape including BCS. For example, the pet may be a dog whose obesity level is determined using a ratio of a waist thickness to a chest thickness.

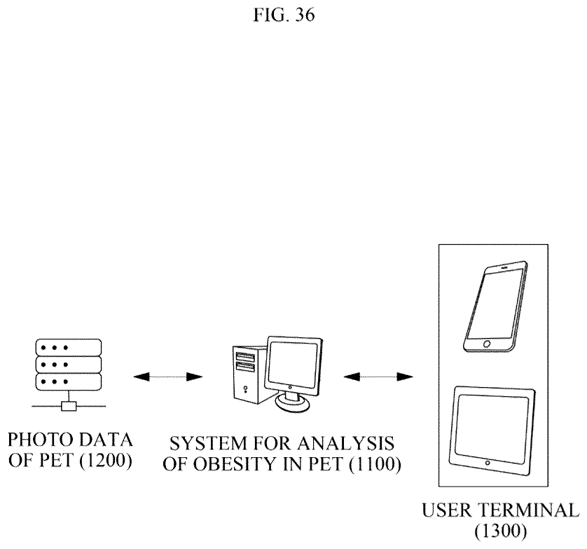

Hereinafter, a configuration and operation of the system for analysis of obesity in a pet according to an embodiment of the present disclosure will be described with reference to FIG. 36.

As illustrated in FIG. 36, the system 1100 for analysis of obesity in a pet according to the present embodiment may include photo data 1200 of the pet and a user terminal 1300. The system 1100 for analysis of obesity in a pet according to another embodiment may be included in the user terminal 1300 that acquires the photo data of the pet.

The photo data 1200 of the pet may be a photo of a pet captured by a user using the user terminal 1300, or may be a photo of a pet uploaded by the user using the user terminal 1300.

The user of the user terminal 1300 may capture a photo so that a body of a targeted pet is clearly visible, and transmit the captured photo data of the pet to the system 1100 for analysis of obesity in a pet. The user of the user terminal 1300 may capture a photo so that the body of the targeted pet is clearly visible, and store the captured pet photo as the photo data 1200 of the pet.

The system 1100 for analysis of obesity in a pet according to another embodiment may include the pre-stored photo data 1200 of the pet. The photo data 1200 of the pet may be the result of capturing the body of the targeted pet so that the body of the targeted pet is clearly visible. The photo data 1200 of the pet pre-stored in the system 1100 for analysis of obesity in a pet may have been uploaded to a separate service server (not illustrated). The separate service server to which the photo data 1200 of the pet is uploaded may provide a signal to a user who suggests a photo guide to upload a photo in which the pet's body is clearly visible.

The user terminal 1300 may have a pet care service application installed therein, with the pet care service application being connected to the system 1100 for analysis of obesity in a pet or a separate service server (not illustrated), and the pet care service application may provide a photo capturing user interface (UI) that guides photo capturing so that the pet's body is clearly visible. Of course, the user terminal 1300 has a web browser installed therein, with the web browser accessing a front-end web page served by the system 1100 for analysis of obesity in a pet or the separate service server (not illustrated), and the front-end web page may provide a photo capturing UI that guides photo capturing so that the pet's body is clearly captured.

The system 1100 for analysis of obesity in a pet, which acquires the photo data of the pet where the body of the targeted pet is clearly visible, may use photo data to determine the pet's body included in the photo data of the pet, determine the body outline, which is a boundary line between a body including hair and a background, and derive body outline flatness. The system 1100 for analyzing obesity information of a pet may determine whether the pet photo is an appropriate photo for determining an obesity level according to the body outline flatness.

The body outline flatness is a value that may be derived using a degree of change in a vector value of the outline when an outline of the pet's body is determined through edge detection of the pet's body and the background included in the photo data of the pet. The body outline flatness is a value that decreases as a distance to move from one point included in the body outline of the pet to another point is longer than a straight distance, and increases as the body outline of the pet approaches a straight line.

The body outline flatness is a value that may be derived through a process of generating a body estimation line of the targeted pet using the body outline, generating a noise vector using the body outline and the body estimation line, and calculating a dispersion representing a diversity of directions of a noise vector.

The body estimation line of the targeted pet may be a line roughly estimated by connecting points that are determined to be close to a position determined to be a center of the pet's body between a plurality of hairs of the pet constituting the body outline.

Using the body estimation line, it is possible to determine the distribution of hair that entirely or partially covers the body estimation line of the body. Since the hair that entirely covers the body estimation line is hair that extends in all directions, the dispersion represented by the noise vector generated by the hair will be more diverse and higher than that of the hair that only partially covers the body estimation line.

The system 1100 for analysis of obesity in a pet may correct the body outline to increase the body outline flatness using the body outline flatness and dispersion. The system 1100 for analysis of obesity in a pet may perform outline correction to increase flatness by determining that it is difficult to derive factors used to determine an obesity level from a body with low body outline flatness, and the system 1100 for analysis of obesity in a pet may perform additional outline correction to increase the flatness by determining that it is more difficult to derive factors used to determine an obesity level from a body with high dispersion.

The system 1100 for analysis of obesity in a pet may determine that it is easy to derive the factors used to determine the obesity level as the body outline flatness included in the photo data exceeds a reference value, and determine that there is no need to reacquire a photo to directly derive the factors used to determine the obesity level.

The system 1100 for analysis of obesity in a pet may determine that it is easy to derive the factors used to determine the obesity level as the body outline flatness included in the photo data is included in a specific section, and derive factors used to partially modify a photo and determine the obesity level from the modified body.

The system 1100 for analysis of obesity in a pet may determine that it is difficult to derive the factors used to determine the obesity level as the body outline flatness included in the photo data is included in the specific section, and may suggest that a user reacquire or change a photo.

The system 1100 for analysis of obesity in a pet, which suggests reacquiring a photo or changing a photo, may display a screen suggesting that a user guides the pet through a specific operation of the pet. The screen on which the system 1100 for analysis of obesity in a pet suggests that the user guides the pet through the specific operation may be a screen of the user terminal 1300. The specific operation of the pet that the system 1100 for analysis of obesity in a pet suggests to the user is an operation of preventing obstruction of hair, which makes it difficult to derive factors used to determine an obesity level due to sagging caused by gravity, and such as making the pet sit or lie down.

The system 1100 for analysis of obesity in a pet may acquire photo data acquired by capturing the pet performing the specific operation, use the acquired photo data to determine a body of the pet included in the photo data of the pet, and derive body outline flatness. The system 1100 for analysis of obesity in a pet may determine whether the pet photo is an appropriate photo for determining the obesity level based on the body outline flatness.

The system 1100 for analysis of obesity in a pet, which suggests reacquiring or changing the photo, may display the screen that suggests recapturing to the user. The screen on which the system 1100 for analysis of obesity in a pet suggests recapturing to the user may be the screen of the user terminal 1300.

The system 1100 for analysis of obesity in a pet, which suggests reacquiring or changing the photo, may display the screen that suggests recapturing to the user. The screen on which the system 1100 for analysis of obesity in a pet suggests moving the capturing device to the user may be the screen of the user terminal 1300. The system 1100 for analysis of obesity in a pet may provide the user with the screen of the terminal that includes graphics displaying a movement line of the capturing device.

The system 1100 for analysis of obesity in a pet may acquire the photo data acquired by capturing a pet from multiple viewpoints, use the acquired photo data to generate a three-dimensional (3D) modeling body of the pet, determine the pet's body included in the 3D modeling body, and derive the body outline flatness. The system 1100 for analysis of obesity in a pet may determine whether the pet photo is an appropriate photo for determining the obesity level based on the body outline flatness.

The system 1100 for analysis of obesity in a pet, which suggests reacquiring the photo or changing the photo, may suggest that the user upload the photo of the pet performing the specific operation or the photo of the pet acquired from multiple viewpoints.

The system 1100 for analysis of obesity in a pet may determine the pet's body included in the reacquired or changed photo data of the pet and derive the body outline flatness. The system 1100 for analysis of obesity in a pet may determine whether the photo of the pet is an appropriate photo for determining the obesity level based on the body outline flatness.

The configuration and operation of the system for analysis of obesity in a pet according to the present embodiment have been described above with reference to FIG. 36. A method of operating the system 1100 for analysis of obesity in a pet according to the present embodiment may be understood in more detail with reference to other embodiments that will be described below. In addition, technical ideas that may be understood through the above-described embodiments of the system for analysis of obesity in a pet according to the present embodiment may be reflected in other embodiments to be described below, even if not specified separately.

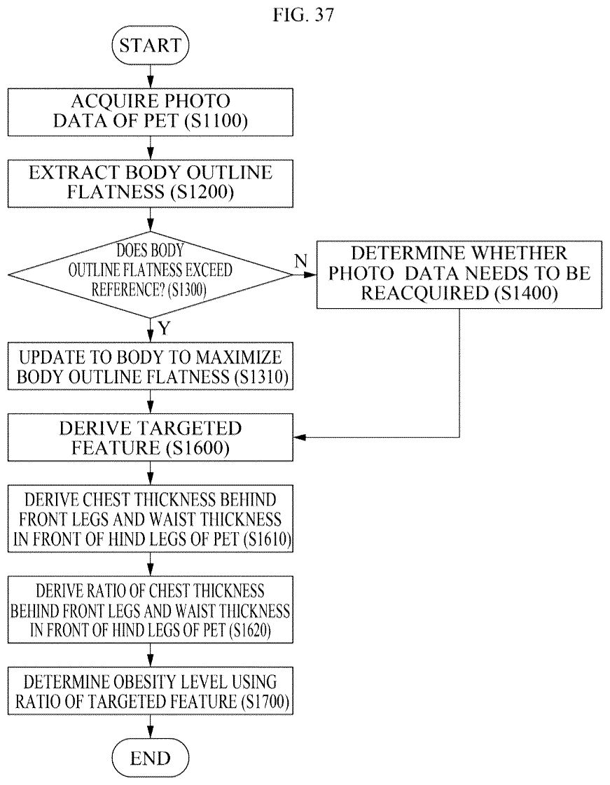

Hereinafter, a method of analyzing obesity information of a pet according to another embodiment of the present disclosure will be described with reference to FIGS. 37 to 39. The method of analyzing obesity information of a pet according to the present embodiment may be performed by a computing device.

Next, a description will be provided with reference to FIG. 37.

In operation S1100, the photo data of the pet may be acquired. As described above, the photo data of the pet may be pre-stored or captured by the user terminal. The photo data of the pet includes the pet's body. The photo data of the pet may include a body between front and hind legs of the pet.

Next, in operation S1200, the body outline flatness may be extracted. The body outline flatness may be determined by the edge detection between the pet's body and the background included in the photo data of the pet acquired in operation S1100, and extracted as the body outline of the pet is determined. The body outline flatness may be derived using the degree of change in a vector value of the outline of the pet's body. The body outline flatness may be a value that decreases as the change in the vector value of the outline of the body increases. The body outline flatness may decrease as a distance to move from one point included in the body outline of the pet to another point is longer than a straight distance, and increase as the body outline of the pet approaches a straight line.

Next, in operation S1300, it may be determined whether the derived body outline flatness exceeds the reference. When the derived body outline flatness exceeds the reference, the body outline of the pet included in the photo is flat, and thus it is determined that the body outline of the pet acquired from the photo is not expanded due to the influence of hair, and the body outline derived without any additional modification process may be reliable.

In operation S1310, the pet's body in the photo data whose derived body outline flatness exceeds the reference may be updated to a body with maximized flatness as some noise is deleted through Gaussian blurring and averaging filters. For example, a sufficiently clear body outline may be inferred, but when a waist thickness corresponding to a feature used to determine an obesity level is derived from a body with hairs partially hanging down at junctions of the legs and abdomen, inaccurate features including noise may be derived. Therefore, it is possible to perform correction to delete partially loose hairs through the Gaussian blurring and averaging filter.

The noise derived from the derived body outline may be a shape formed by hairs with different lengths compared to some surrounding areas, which reduces the body outline flatness.

Next, in operation S1600, a targeted feature may be derived from the body with maximized flatness. The targeted feature may be the chest thickness behind the front legs of the pet and the waist thickness in front of the hind legs of the pet derived in operation S1610. In operation S1620, the ratio between the chest thickness behind the front legs of the pet and the waist thickness in front of the hind legs of the pet may be derived.

Next, in operation S1700, the obesity level may be determined using the ratio of the chest thickness behind the front legs of the pet and the waist thickness in front of the hind legs of the pet. In general, the waist of a pet with normal weight should be a certain degree thinner than its chest, and therefore the ratio of the chest thickness behind the front legs of the pet and the waist thickness in front of the hind legs of the pet may be a factor from which the obesity level of the pet is determined.

In operation S1300, when the derived body outline flatness is less than or equal to the reference, it is determined that the body outline of the pet included in the photo is not flat and the body outline of the pet acquired in the photo is expanded due to the influence of hair, and it may be determined in operation S1400 whether the separate photo data needs to be reacquired.

When the separate photo data is reacquired, the process after operation S1400 in which the targeted feature is derived from the reacquired data may be repeated.

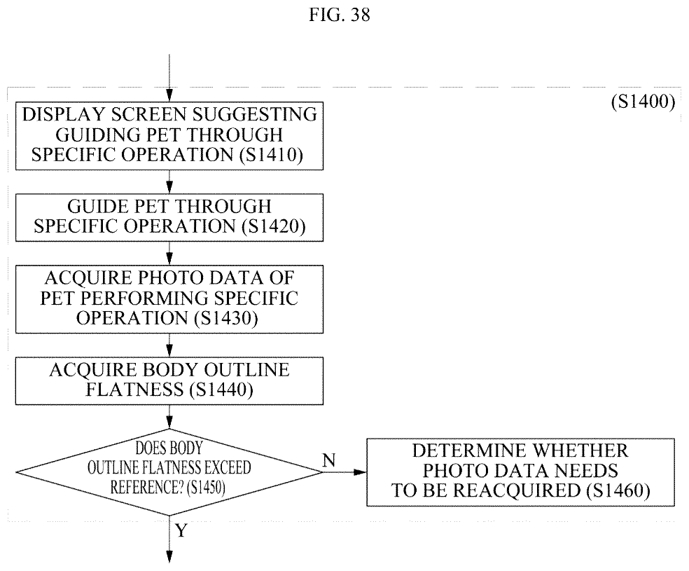

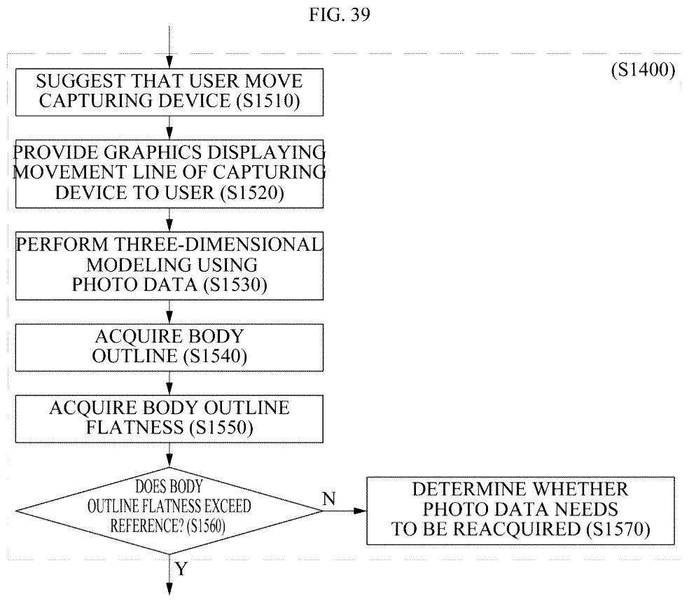

Hereinafter, the operation S1400 of determining whether the separate photo data needs to be reacquired will be described in more detail with reference to FIGS. 38 and 39.

In operation S1410, the screen suggesting the user guides the pet through the specific operation may be displayed. The guidance of the pet through the specific operation may be determined when the body outline flatness derived in operation S1300 is included in a specific section. In operation S1420, a user may group the specific operation displayed on the screen. The specific operation through which the pet is guided may include an operation of sitting, lying down, or wetting its abdomen with water. The specific operation through which the pet is guided by the user may be determined according to the section including the derived body outline flatness. Depending on the degree of body outline flatness, the method of hanging down hair of a pet with hair may vary.

The pet's body included in the photo data of the pet performing the specific operation through which the user guides it acquired in operation S1430 may be a body to which hair hanging down by gravity is attached. The body outline flatness acquired in operation S1440S may be the flatness acquired from the body outline of the pet, which is the body to which the hair hanging down by gravity is attached.

Next, in operation S1450, it may be determined again whether the flatness acquired from the body outline of the pet with hair attached to the body exceeds the reference value. When the flatness acquired from the body outline of the pet with hair attached to the body is less than or equal to the reference value, as it is determined in operation S1460 that the operation taken in operation S1400 did not match the hair type of the targeted pet, it is determined whether the photo data needs to be reacquired again, and the operation S1410 of displaying the screen suggesting guiding the pet through the specific operation may be repeated.

When the flatness acquired from the body outline of the pet with hair attached to the body exceeds the reference value, as it is determined in operation S1460 that the operation taken in the operation S1400 matches the hair type of the targeted pet, it may proceed to the next operation to determine the obesity level from the body outline.

When the body outline flatness derived in operation S1300 is less than or equal to a certain reference, it may be determined to perform the 3D modeling using the photo data. In order to acquire the data to be used for the 3D modeling, in operation 1510S11510, the screen suggesting moving the capturing device may be displayed to the user. In operation S1520, the screen including graphics displaying the movement line may be provided as the screen suggesting moving the capturing device to the user.

Next, the photo data acquired by the user along the movement line may be 3D modeled in operation S1530. The 3D modeled pet body data may be acquired in operation S1540. The 3D modeled pet body data may be body data modeled by connecting surfaces that are partially exposed between fluffy hairs of the pet and obscured in a single photo acquired in one direction. For the pet with the fluffy hair, the hair may not lie on the body even when the pet lies down or sits down.

Therefore, the body outline flatness, which is determined to be used for the 3D modeling using the photo data, may indicate that the acquired outline flatness is less than or equal to the predefined reference and the dispersion representing the diversity of directions of the noise vector used when deriving the flatness is determined to be the reference value or greater.

The noise vector is a vector extending foreign substances from the center of the body to the outside, with the foreign substances lowering the flatness of hair, etc., and generating noise based on the body estimation line which is a roughly estimated line connecting points determined to be close to the center of the pet's body between multiple hairs constituting the body outline. Higher dispersion representing the diversity of directions of the noise vector may indicate that hair is growing in different directions.

Next, in operation S1550, the body outline flatness may be acquired from the pet's body acquired by the 3D model. Next, in operation S1560, it may be determined again whether the flatness of the body outline acquired from the pet's body acquired by the 3D model exceeds the reference value.

When the flatness of the body outline acquired from the pet's body acquired by the 3D model is less than or equal to the reference value, it may be determined in operation S1570 that the photo data acquired by the user in operations 1510S11510 and S1520 was inappropriate data for determining the obesity level. Accordingly, operation 1510S11510 of determining whether the photo data needs to be reacquired and displaying the screen suggesting moving the capturing device to the user may be repeated.

When the flatness of the body outline acquired from the pet's body acquired by the 3D model exceeds the reference value, it may be determined in operation S1570 that the photo data acquired by the user in operations 1510S11510 and S1520 was appropriate data for determining the obesity level. Accordingly, it may proceed to the next operation to determine the obesity level from the body outline.

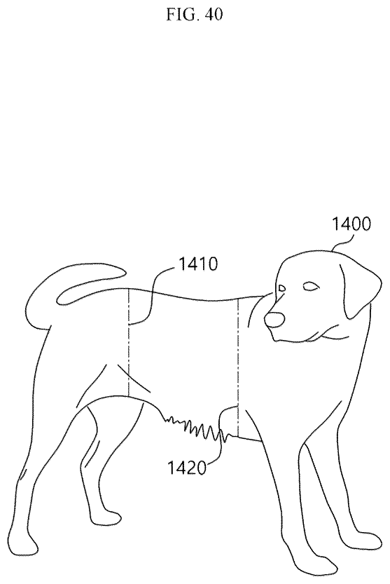

Next, a method of determining obesity in a pet will be described with reference to FIG. 40. The targeted pet 400 illustrated in FIG. 40 is the pet's body included in the photo data of the pet, and FIG. 40 clearly illustrates a thinnest waist thickness 410 in front of the hind legs and a thickest chest thickness 420 behind the front legs.

Generally, when extracting the waist thickness in front of the hind legs of the pet and the chest thickness behind the front legs of the pet, the hair that hangs down under the abdomen due to gravity may obscure the exact waist and chest thickness. A silhouette from the waist to the chest of the targeted pet 400 illustrated in the drawing may be sufficiently extracted from the photo data.

When extracting the waist thickness 410 in front of the hind legs and the chest thickness 420 behind the front legs of the targeted pet 400 illustrated in the drawing, the hair that hangs down under the abdomen due to gravity may not affect the extraction of the accurate waist thickness, but may cause confusion when extracting the chest thickness.

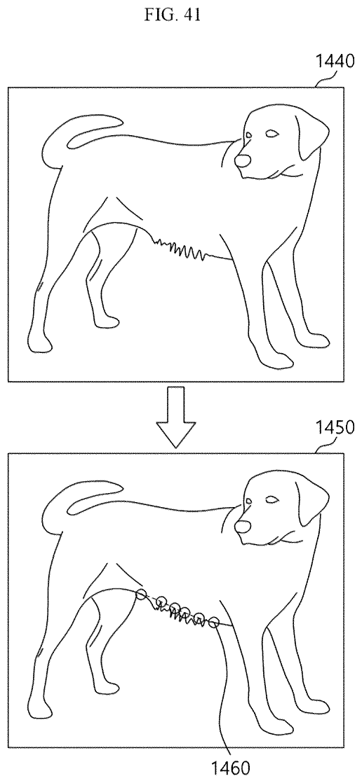

Therefore, a method of removing some noise by applying the Gaussian blurring and averaging filters to the pet's body in the photo data whose body outline flatness derived with reference to FIG. 41 exceeds the reference and updating the body with maximized flatness into the pet's body will be described.

Referring to the photo data 440 before the update, the distance moving from one point included in the body outline of the pet to another point in the pet's body included in the photo data 440 is not longer than the predefined reference compared to the straight distance. As the body outline of the pet is close to a straight line, it may be determined that the outline flatness of the pet's body included in the photo data 440 exceeds the predefined reference.

Since the body outline flatness of the pet in the pet's body included in the photo data 440 exceeds the predefined reference, a sufficiently clear body outline may be inferred, but when deriving the waist thickness corresponding to the feature used to determine an obesity level from a body with hairs partially hanging down at the junctions of the legs and abdomen, inaccurate features including noise may be derived.

Therefore, correction may be performed to delete partially hanging hairs or derive a body outline without hairs to increase the body outline flatness through the Gaussian blurring and averaging filter. Referring to the photo data 450 from which the correction point is derived, a point between hairs closer to the body compared to the hanging hairs may be designated as the correction point 460.

When a point between hairs is designated as the correction point 460, the body using the line connecting the correction point 460 as the body outline may be updated to the pet's body. When extracting the waist thickness in front of the hind legs of the pet and the chest thickness behind the front legs of the pet using the photo data including the pet's body having the updated body outline, it is possible to avoid being disturbed by the hair that hangs down under the abdomen due to gravity and determine a more accurate obesity level.

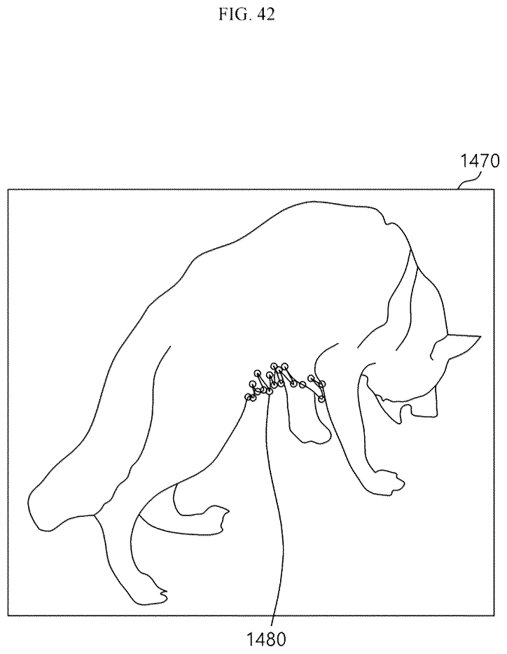

Next, the photo data from which it is difficult to determine the obesity level using only the pet's body included in the photo data will be described with reference to FIG. 42.

Referring to photo data 470 illustrated in FIG. 42, the distance moving from one point included in the body outline of the pet to another point on the pet's body included in the photo data 470 is longer than the predefined reference compared to the straight line distance. As the sequential relationship between points 1480 of the body outline is unclear when connecting the body outline of the pet, it may be determined that the outline flatness of the pet's body included in the photo data 470 is less than the predefined reference. Therefore, since it is difficult to determine the obesity level only with the pet's body included in the photo data 470, it may be determined that the photo data needs to be reacquired.

It may be confirmed that the noise vector of the surface noise used when deriving the body outline flatness of the pet from the pet's body included in the photo data 470 of FIG. 42 is less than a predefined level. In order for the noise vector to be greater than or equal to the predefined level, the outline whose flatness is less than the reference value should be acquired on the ventral side and a dorsal side which is an opposite side of a torso. Therefore, the dispersion, which represents the diversity of directions of the noise vector used to derive the body outline flatness of the pet extracted from the pet's body included in the photo data 470, may be less than the reference value.

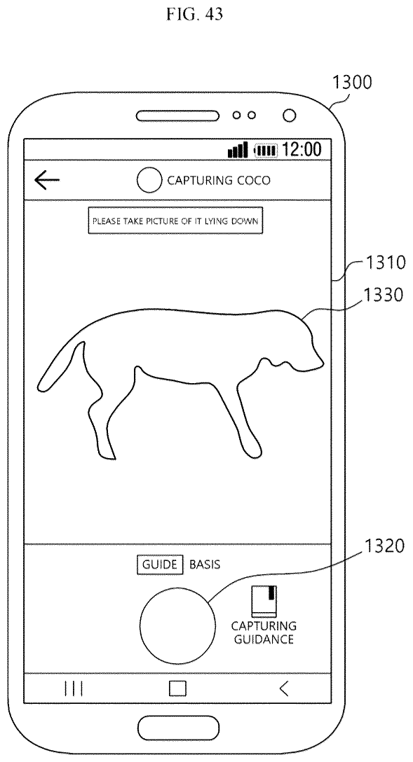

As it is determined that the dispersion, which represents the diversity of directions of the noise vector used to derive the body outline flatness, is less than the reference value, the reacquisition of the photo data may be the reacquisition of the photo data of the pet performing the specific operation. A method of reacquiring photo data by guiding a pet through a specific operation will be described below with reference to FIG. 43.

FIG. 43 illustrates the user terminal 1300 on which a screen 310 suggesting guiding a pet through a specific operation is displayed. The screen suggesting guiding the pet through the specific operation may be provided to a user when the body outline flatness of the pet is included in a specific section. The screen suggesting guiding the pet through the specific operation may include an example guide 330 of an operation through which to guide the pet. The screen suggesting guiding the pet through the specific operation may receive a capture from a user using a photo data acquisition button 133011330 or automatically perform the capture when the pet is included in the example guide 330 of the action through which to guide the pet to acquire the photo data at the relevant point in time.

The specific operation through which the pet is guided may include an operation of sitting, lying down, or wetting its abdomen with water. The pet's body included in the photo data of the pet performing the specific operation may be a body with hairs hanging down due to gravity attached to the body. The body outline flatness may be the flatness acquired from the body outline of the pet, which is the body on which the hair hanging down due to gravity lies.

It may be determined again whether the flatness acquired from the body outline of the pet with hair lying on the body is less than the reference value. When the flatness acquired from the body outline of the pet with hair lying on the body is less than the reference value, as it is determined that a current operation performed by the pet did not match the hair type of the targeted pet, it is determined whether the photo data needs to be reacquired again, and the operation of displaying the screen suggesting guiding the pet through the specific operation may be repeated.

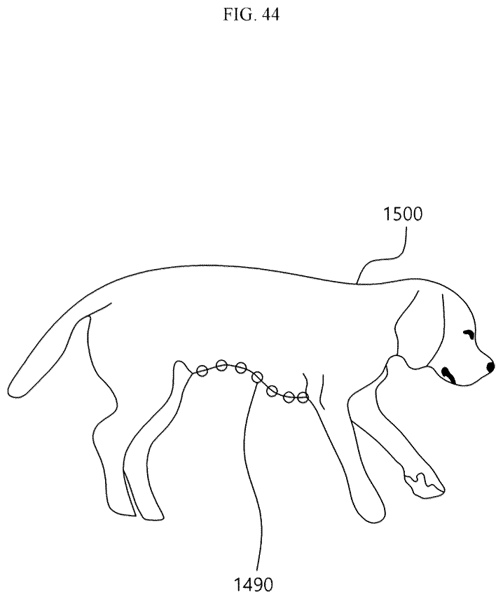

FIG. 44 illustrates a pet's body 500 included in the photo data of the pet performing the specific operation. When the pet performs an operation that makes the pet's hair stand up, the hair may hang down to the floor due to gravity and cause confusion in measuring the thicknesses of the abdomen, waist, and chest, and when the pet performs a lying down operation as illustrated with the pet's body 500, the hair may hang down to the side and not interfere with the measurement of the thicknesses of the abdomen, waist, and chest. It may be determined that the body outline flatness generated by connecting outline points 1490 of the pet's body 500 with hair hanging down to the side by performing the lying down operation exceeds a predefined reference.

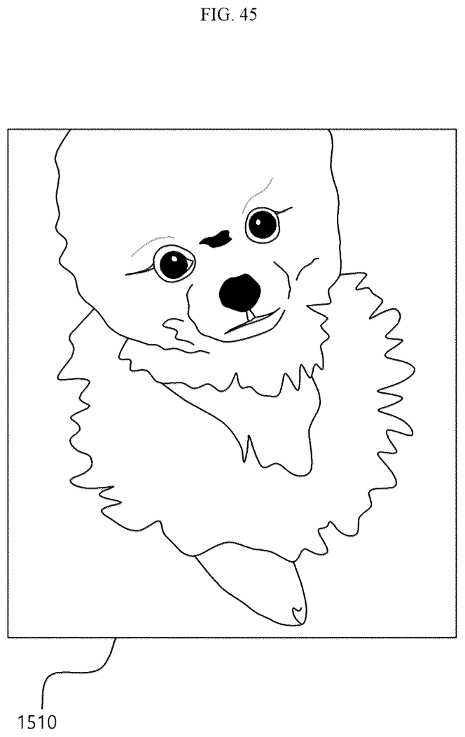

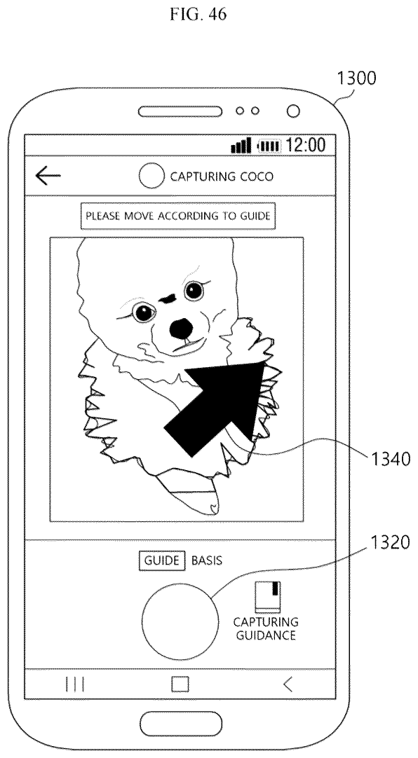

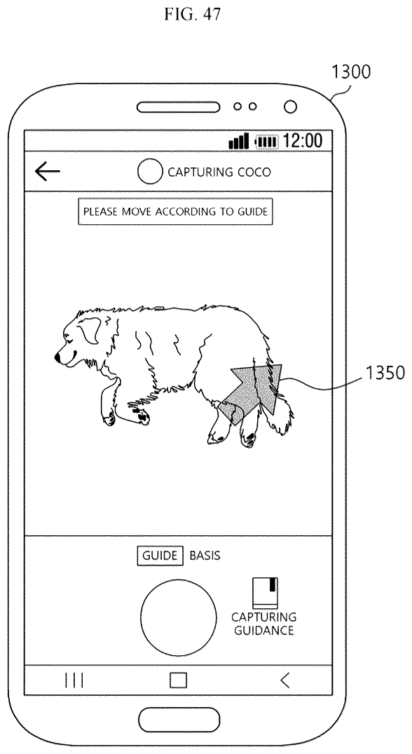

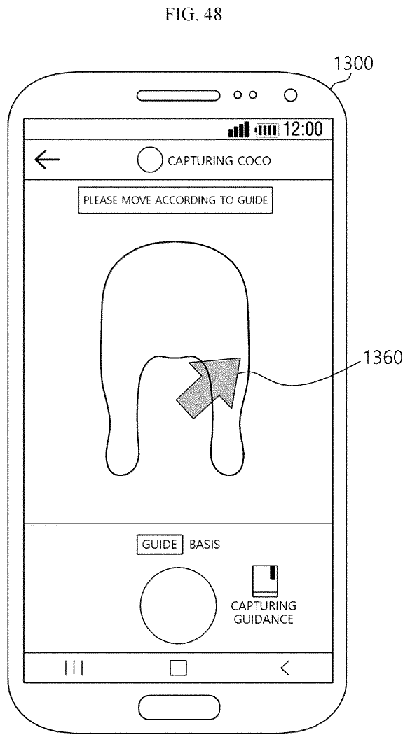

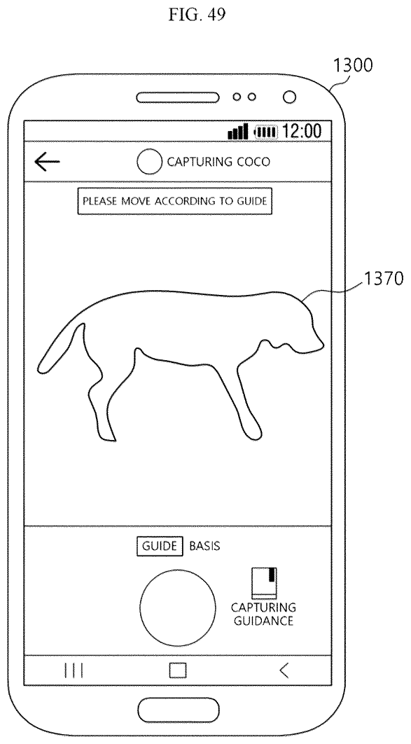

Next, FIG. 45 illustrates photo data 1510 including a pet's body with an outline whose flatness is less than the reference value on the ventral side and the dorsal side which is the opposite side of the body. FIG. 45 illustrates that the flatness on each of the ventral side of the pet and the dorsal side of the pet, which is the opposite side of the torso, included in the photo data 1510 is less than the reference value, and the dispersion represented by the noise vector is greater than or equal to a predefined level. When the body outline flatness is less than the reference value and the dispersion represented by the noise vector is high, the reacquisition of the photo data may be the reacquisition of the photo data to be used for generating the 3D modeling body. A method of reacquiring data to be used to generate a 3D modeling body of a pet will be described below with reference to FIGS. 46 to 49.

FIGS. 46 to 49 illustrate the user terminal 1300 displaying a screen suggesting capturing a pet in a specific direction. A pet may be captured from multiple viewpoints by a user who moves a camera and performs capturing according to the screen suggesting capturing the pet in a specific direction, and photo data may be acquired. The 3D modeling body of the pet may be generated using the acquired photo data. When the pet's body included in the 3D modeling body is determined, the body outline flatness of the targeted pet may be derived.

The screen suggesting capturing the pet in the specific direction may include direction arrows 1340, 1350, and 1360 or a direction example guide 1370 as the graphics displaying the capturing device movement line.

The direction arrows 1340, 1350, and 1360 may be graphic objects that intuitively display the direction in which the capturing device should move based on the pet. The direction arrows 1340, 1350, and 1360 may be graphic objects suggesting capturing the photo data necessary to acquire a skin surface under the pet's hair. The direction arrows 1340, 1350, and 1360 may be graphic objects that designate and point to a local area to capture an area determined as a shade of hair. The direction arrows 1340, 1350, and 1360 may suggest capturing a back of the pet, for example. When capturing at that location, it is possible to acquire points between hairs to distinguish the skin surface under the pet's hair and the hair, and generate a body using the line connecting the points between the hairs as the body outline.

The direction example guide 1370 may be a graphic object that displays an example body of a pet that should be visible in the direction in which the capturing device should move. The direction example guide 1370 may be a graphic object that suggests capturing necessary photo data by illustratively displaying a direction in which to capture the pet, such as left, right, rear, top, and bottom. The direction example guide 1370 may suggest a specific pose of a pet and suggest capturing in a specific direction. For example, it may suggest capturing a back of a sitting pet. When capturing at that location, it is possible to acquire points between hairs to distinguish the skin surface under the pet's hair and the hair, and generate a body using the line connecting the points between the hairs as the body outline.

So far, the method of analysis of obesity in a pet according to some embodiments of the present disclosure has been described in detail. The embodiments described above are illustrative in all respects and should be understood as non-limiting.

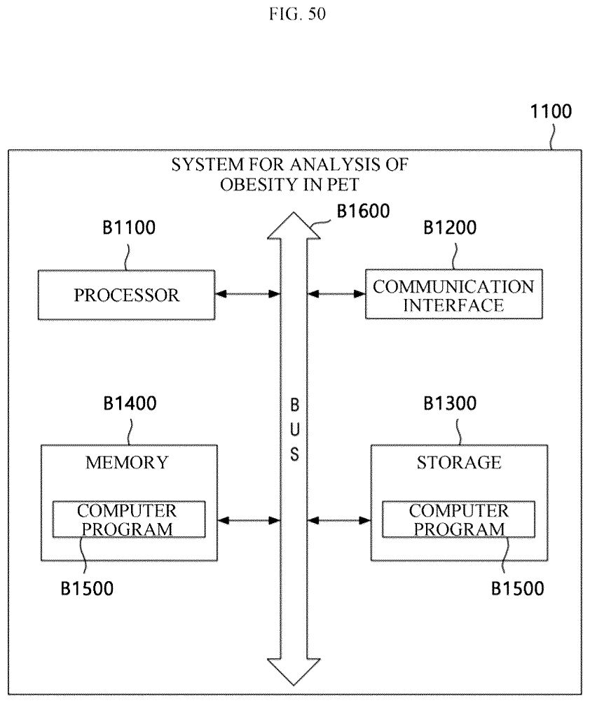

FIG. 50 is a hardware configuration diagram of a system for analysis of obesity in a pet according to some embodiments of the present disclosure. The system 1100 for analysis of obesity in a pet in FIG. 50 includes one or more processors B1100, a system bus B1600, a communication interface B1200, a memory B1400 into which the computer program B1500 executed by the processor B1100 is loaded, and a storage B1300 that stores the computer program B1500.

The processor B1100 controls the overall operation of each component of the system 1100 for analysis of obesity in a pet. The processor B1100 may perform operations on at least one application or program to execute methods/operations according to various embodiments of the present disclosure. The memory B1400 stores various types of data, commands and/or information. The memory B1400 may load one or more computer programs B1500 from the storage B1300 to execute methods/operations according to various embodiments of the present disclosure. The bus B1600 provides a communication function between components of the system 1100 for analysis of obesity in a pet. The communication interface B1200 supports Internet communication of the system 1100 for analysis of obesity in a pet. The storage B1300 may non-transitorily store one or more computer programs B1500. The computer program B1500 may include one or more instructions implementing methods/operations according to various embodiments of the present disclosure. When the computer program B1500 is loaded into the memory B1400, the processor B1100 may perform the methods/operations according to various embodiments of the present disclosure by executing the one or more instructions.

In some embodiments, the system 1100 for analysis of obesity in a pet described with reference to FIG. 50 may be configured using one or more physical servers included in a server farm based on cloud technology such as a virtual machine. In this case, at least some of the processor B1100, the memory B1400, and the storage B1300 among the components illustrated in FIG. 50 may be virtual hardware, and the communication interface B1200 may also be composed of virtualized networking elements such as a virtual switch.

The computer program B1500 may include an instruction to acquire photo data of the pet; an instruction to derive body outline flatness of the pet using the photo data, an instruction to determine whether the photo data needs to be reacquired using the body outline flatness when it is determined that the photo data needs to be reacquired, an instruction to display a photo recapturing screen including a guide for changing the pet's posture, and when it is determined that the photo data does not need to be reacquired, an instruction to determine the obesity of the pet by analyzing the photo data.
[3]

Even for pets, teeth are also one of the important body parts that determine the health of the pets. In particular, dogs have a total of 42 teeth. Since dogs have many teeth, they have a wide range of teeth brushing, and unlike humans, have a deep oral structure, so it is easy for dogs to develop tartar, making it difficult to maintain healthy teeth.

Currently, a dental health status of pets is measured through direct examination by experts using X-ray data, etc.

Meanwhile, diagnostic assistance technologies based on artificial intelligence technology are provided. A representative example is machine learning-based artificial intelligence that predicts the likelihood of developing a specific disease or its severity by analyzing photos of problem areas. In situations where it is difficult to visit a veterinary hospital, artificial intelligence is provided to predict the likelihood of developing a specific disease or its severity by analyzing images of targeted pets taken by a pet's guardian using a smartphone, etc.

However, basic and elemental technologies for photographing teeth in pets with a terminal and inferring dental health in pets using the artificial intelligence did not exist in Korea, and there was little discussion on the use of such artificial intelligence.

Hereinafter, various embodiments of the present disclosure will be described in detail with reference to the accompanying drawings.

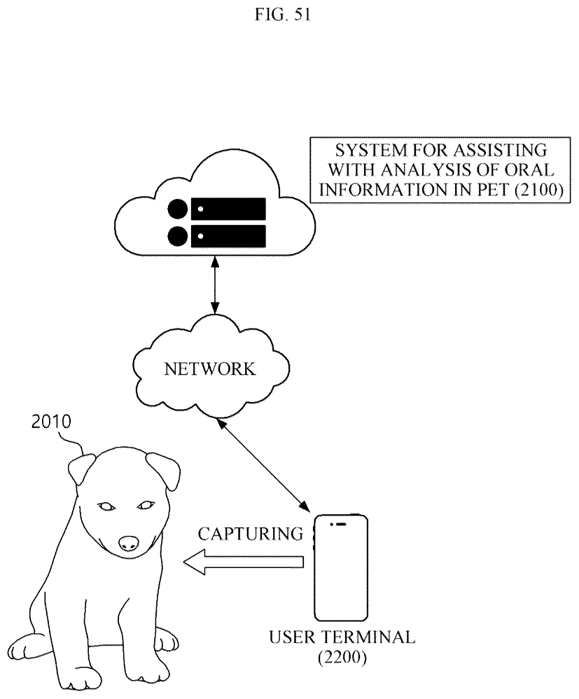

First, a configuration and operation of a system for analysis of oral information in a pet according to an embodiment of the present disclosure will be described with reference to FIG. 51.

As illustrated in FIG. 51, the system for analysis of oral information in a pet according to the embodiment may include a system 2100 for assisting with analysis of oral information of a pet and a user terminal 2200. A user of the user terminal 2200 may capture a photo so that teeth, crown color, and a gum line of a targeted pet are clearly visible, and transmit a captured image, which is the result of the capturing, to the system 2100 for assisting with analysis of oral information of a pet.

The user terminal 2200 may be installed with a pet oral information analysis application connected to the system 2100 for assisting with analysis of oral information of a pet or a separate service server, and the pet oral information analysis application may provide a photo capturing user interface (UI) that guides capturing a photo so that the color and gum line of the pet's teeth and crowns are clearly visible.

Of course, a web browser that accesses the system 2100 for assisting with analysis of oral information of a pet or a front-end web page served by the separate service server is installed in the user terminal 2200, and the front-end web page may provide the photo capturing UI that guides capturing the photo so that the color and gum line of the pet's teeth and crowns are clearly visible.

In addition, the web browser that accesses the system 2100 for assisting with analysis of oral information of a pet or the front-end web page served by the separate service server is installed in the user terminal 2200, and the front-end web page may provide, to the server, the photo capturing UI that guides capturing the photo so that the color and gum line of the pet's teeth and crowns are clearly visible. The results analyzed by the server that received the photo may be received again by the user terminal 2200, and the user terminal 2200 may provide the UI that provides the results to the user.

The system 2100 for assisting with analysis of oral information of a pet may refer to a virtual machine or a container provisioned on one or more cloud nodes. Of course, the system 2100 for assisting with analysis of oral information of a pet may be implemented through one or more on-premise physical servers. The system 2100 for assisting with analysis of oral information of a pet may store definition data of a pet gum line scoring model previously generated through machine learning. A machine learning runtime (ML runtime), which is software for executing the pet gum line scoring model, may be installed. That is, the system 2100 for assisting with analysis of oral information of a pet may receive a captured oral image of the pet from the user terminal 2200, input the data of the acquired captured oral image to the gum line scoring model, generate oral information analysis results using the data output from the gum line scoring model, and transmit data including the generated oral information analysis results to the user terminal 2200. The oral information analysis results include information from which the crowns and gingiva of the teeth segmented into individual teeth and a boundary lines which are boundaries between the crowns and the gingiva can be derived.

In some embodiments, the system 2100 for assisting with analysis of oral information of a pet may perform machine learning on the gum line scoring model periodically or aperiodically and deploy the updated gum line scoring model to the user terminal 2200. That is, in this case, the system 2100 for assisting with analysis of oral information of a pet may serve as a device that executes a machine learning pipeline of the gum line scoring model, and the user terminal 2200 may serve as a type of edge device that executes the machine learning application.

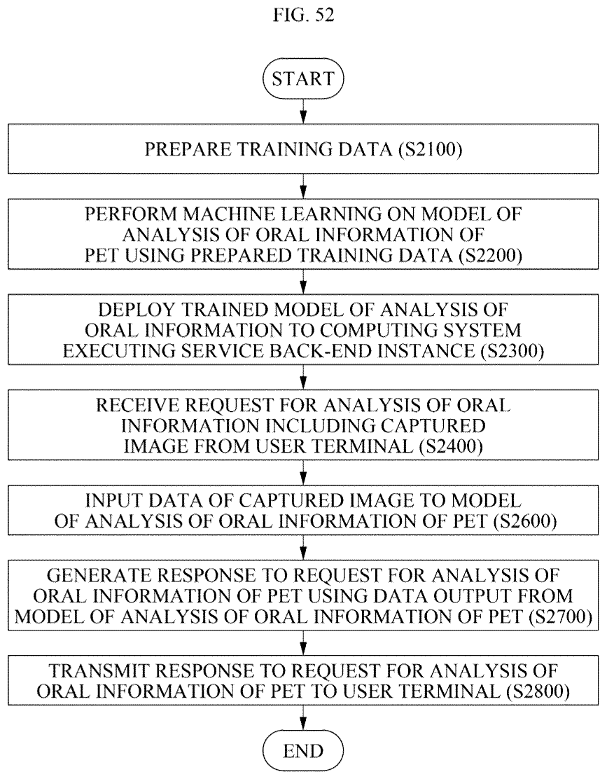

The method of analysis of oral information in a pet according to another embodiment of the present disclosure will be described with reference to FIG. 52. The analysis of oral information of a pet according to the present embodiment may be performed by one or more computing devices. That is, in the method of analysis of oral information in a pet according to the present embodiment, all operations may be performed by one computing device, or some operations may be performed by another computing device. For example, some operations may be performed by a first server system and other operations may be performed by a second server system.

In addition, as the server system is implemented on a cloud computing node, operations performed by one server system may also be performed separately on a plurality of cloud computing nodes. Hereinafter, in describing the method of analysis of oral information in a pet according to the present embodiment, description of the performance subject of some operations may be omitted, and in this case, the performance subject may be the system 2100 for assisting with analysis of oral information of a pet described with reference to FIG. 51.

In operation S2100, training data is prepared. The training data may include a plurality of photos captured so that the color and gum line of the pet's teeth and crowns are clearly visible, and labeling data for each photo.

The labeling data may include, for example, whether periodontitis occurs for each photo and one or more pieces of additional information. For example, the additional information may include information such as the pet's age and the latest scaling date.

The labeling data may be composed only of whether periodontitis occurs for each photo, for example.

In operation S2200, a model of analysis of oral information of a pet is machine-learned using the prepared training data. By automating the training data acquisition and establishing a metric of the training data acquisition situation, the machine learning on the model of analysis of oral information of the pet may be repeatedly performed periodically or aperiodically, and the timing of performing such machine learning may be determined based on the value of the metric.

Next, when the machine learning is completed, in operation S2300, the trained model of analysis of oral information may be deployed to a computing system executing a service back-end instance. As described above, the computing system executing the back-end instance may be a service server.

When a request for analysis of oral information including a captured image is received from the user terminal in operation S2400, the data of the captured image may be input to the model of analysis of oral information of the pet in operation S2600. In this case, of course, pre-designated pre-processing, such as cropping, resizing, or applying a color filter, may be performed on the captured image.

In operation S2700, a response to the request for analysis of oral information of the pet may be generated using the data output from the model of analysis of oral information, and in operation S2800, the response to the request for analysis of oral information of the pet may be transmitted to the user terminal.

According to the embodiment, a pet's guardian may confirm analysis results related to oral information simply by capturing an oral cavity image of a pet. For example, information on the occurrence and progress of periodontitis may be displayed on the user terminal.

For a more accurate analysis of oral information of a pet, the unique artificial neural network architecture of the present disclosure, which allows features that are reflected in a pet's oral image to improve the accuracy of information analysis to be well reflected in the pet's oral image, will be described below.

The artificial neural network architecture of the model of analysis of oral information described below is elaborately designed to identify periodontitis that may occur in the pet's oral cavity as accurately as possible by integrating information from various perspectives in the model of analysis of oral information. Hereinafter, examples of the artificial neural network architecture of the model of analysis of oral information that analyzes the pet's captured oral image will be described with reference to FIGS. 3 to 7.

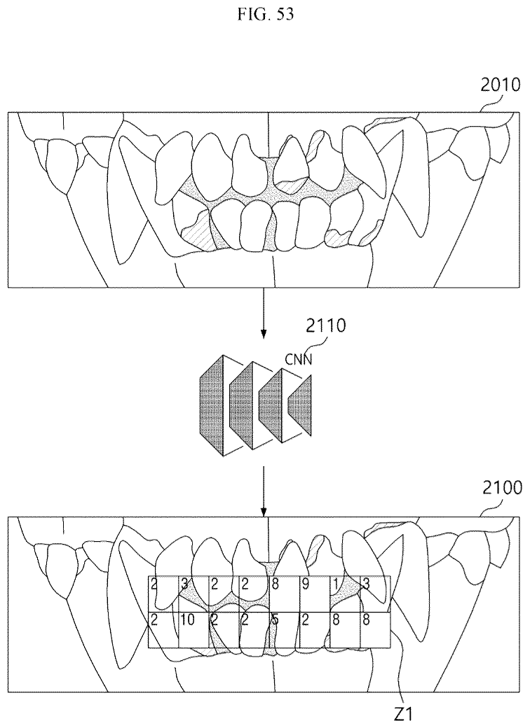

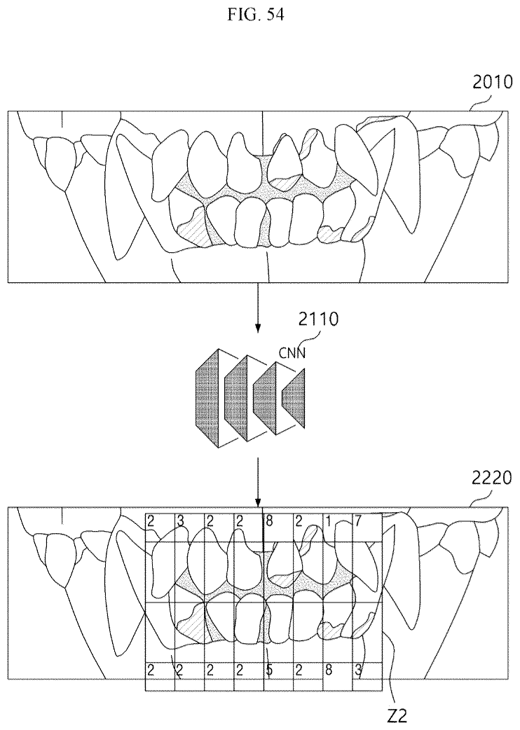

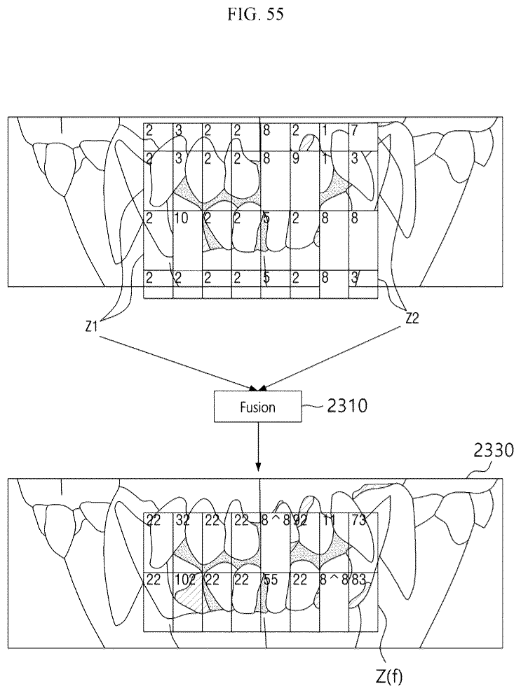

A CNN 2110 that receives the captured image 2010 and outputs a score Z1 obtained by scoring information on color uniformity of each segmented crown area will be described with reference to FIG. 53. The CNN 2110 that receives the captured image 2010 may output information on the color uniformity of each crown area as a scoring score.

The crown area is a visible part of teeth and determines the shape and color of the teeth. The crown area is used to evaluate the size, shape, alignment, etc., of the teeth, and provides important information in dental care.

The color uniformity of the crown area is a reference that indicates whether colors of pixels that constitute the crown area in the captured image are consistent and uniform, and means that pixels within the crown area have similar colors, and the smaller the color difference, the more uniform the color.

The CNN 2110 may output a result of scoring the degree of color uniformity using a color uniformity analysis model generated by performing training with the normal dog image.

The CNN 2110 may output a higher score as the color uniformity of one targeted crown area is extreme. The extreme color uniformity means that color is not uniform. In other words, from a high color uniformity score, it may be determined that the farther away the colors included in one targeted crown area based on a red, green, and blue (RGB) code, the more extreme the color uniformity of the targeted crown area. It may be determined that the closer the area with distant colors based on the RGB code included in one targeted crown area, the more extreme the color uniformity of the targeted crown area. The more extreme the color uniformity of the targeted crown area and the higher the score, the higher the probability that inflammation-related symptoms are present.

In an image 2210 in which the score Z1 on the information on the color uniformity of the crown area is overlaid on the captured image 2010, the more extreme the color uniformity, the higher a score may be output.

The CNN 2110 that receives the captured image 2010 and outputs a score Z2 obtained by scoring the information on the color uniformity of each segmented gingival area will be described with reference to FIG. 54. The CNN 2110 that receives the captured image 2010 may output the information on the color uniformity of the gingival area as a scoring score.

The gingival area is gum parts adjacent to the teeth. The gingival determines the color and tissue condition of the gums, and the health condition of the gingival area has a significant impact on oral hygiene and dental care.

The color uniformity of the gingival area is a reference that indicates whether colors of pixels that constitute the gingival area in the captured image are consistent and uniform, and means that pixels within the gingival area have similar colors, and the smaller the color difference, the more uniform the color.

The CNN 2110 may output a result of scoring the degree of color uniformity using a color uniformity analysis model generated by performing training with the normal dog image.

The CNN 2110 may output a higher score as the color uniformity of one targeted gingival area is extreme. It may be determined that the farther away the colors included in one targeted gingival area based on the RGB code, the more extreme the color uniformity of the targeted gingival area. It may be determined that the closer the area with distant colors based on the RGB code included in one targeted gingival area, the more extreme the color uniformity of the targeted gingival area. The more extreme the color uniformity of the targeted gingival area and the higher the score are, the higher the probability that inflammation-related symptoms are present.

In an image 2220 in which the score Z2 on the information on the color uniformity of the gingival area is overlaid on the captured image 2010, the more extreme the color uniformity, the higher a score may be output.

FIG. 55 illustrates an image 2230 overlaid with a fusion score Z(f) output as a result of a feature fusion logic 2310 that receives the score Z1, which scores the information on the color uniformity of the crown area and the score Z2, which scores the information on the color uniformity of the gingival area by the CNN.

The information on the color uniformity of the crown area and the color uniformity of the gingival area, which is the targets of feature fusion, is information that may affect the probability that the inflammation-related information is present in the oral cavity.

Specifically, the further away the colors included in one target crown or gingival area based on the RGB code, or the more similar the area with the distant color based on the included RGB code, the higher the probability that the inflammation-related symptoms are present.

The fact that the colors included in one targeted crown area are far apart based on the RGB code may mean that tartar has accumulated on the crown, corrosion has occurred, cavities have occurred, or foreign substances are present on the crown. The fact that the colors included in one targeted gingival area are distant based on the RGB code may mean that the gingiva is swollen, bleeding has occurred in the gingiva, or foreign substances are present in the gingiva.

The fact that the areas with distant colors are similar based on the RGB code included in one targeted crown area may mean that tartar is excessively accumulated on the crown or that cavities have progressed significantly. The fact that the areas with distant colors are similar based on the RGB code included in one targeted gingival area may mean that the degree of swelling of the gingiva may be severe or excessive bleeding may have occurred in the gingiva.

As described above, the information affecting the color uniformity of the crown or gingival area may be generated by the presence of the inflammatory symptoms in the oral cavity, generated by temporary problems occurring in individual objects, or generated by the presence of foreign substances.

Therefore, the score Z1 on the information on the color uniformity of the crown area and the score Z2 on the information on the color uniformity of the gingival area of the same tooth as the crown area may be input to the feature fusion logic 2310, and the fusion score Z(f) may be output from the feature fusion logic 2310.

When the color uniformity of the crown area and the color uniformity of the gingival area of the same tooth as the crown area are each greater than a predefined score, and the color uniformity of the crown area and the color uniformity of the gingival area have a difference less than or equal to the reference value, the fusion score Z(f) of the corresponding tooth may be augmented according to a predefined method. The method in which the fusion score Z(f) of the tooth is augmented according to the predefined method may include a method of squaring the fusion score Z(f) of the tooth or a method of assigning a weighted score. The above weighted score may be a score that may not be derived using the color uniformity of the crown area or the color uniformity of the gingival area.

When the color uniformity of the crown area and the color uniformity of the gingival area of the same tooth as the crown area are each greater than the predefined score, and the difference between the color uniformity of the crown area and the color uniformity of the gingival area exceeds the reference value, a temporary individual problem may have occurred in either the crown area or the gingival area, or foreign substances may be present.

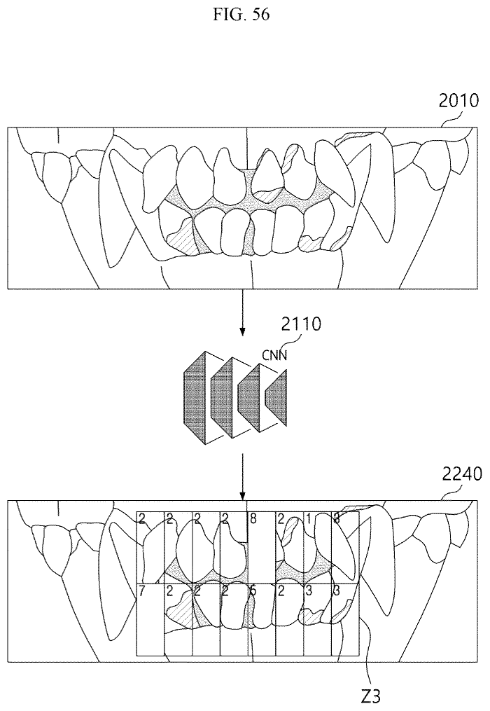

In addition, although not illustrated, the target of the feature fusion may be the information on the color uniformity of the gingival area and the information on the degree of recession of the gum line. The information on the color uniformity of the gingival area and the information on the degree of recession of the gum line are information that may affect the probability that the inflammation-related symptoms are present in the oral cavity. The information on the color uniformity of the gingival area and the information on the degree of recession of the gum line are information that reflects the health condition of the gum. The information on the degree of recession of the gum line will be described later with reference to FIG. 56.

Specifically, the further away the colors included in the targeted gingival area based on the RGB code, or the more similar the area with distant colors based on the included RGB code, or the more severe the degree of recession of the gum line, the higher the probability that the inflammation-related symptoms are present.

The fact that the colors included in one targeted gingival area are distant based on the RGB code may mean that the gingiva is swollen, bleeding has occurred in the gingiva, or foreign substances are present in the gingiva. The fact that the areas with distant colors are similar based on the RGB code included in one targeted gingival area may mean that the degree of swelling of the gingiva may be severe or excessive bleeding may have occurred in the gingiva. The recession in one target gum line may be due to a physical accident occurring to the gum, leaving behind a scar. The gum line may differ depending on the oral structure of each individual.

As described above, the information affecting the color uniformity of the gingival area may be generated by the presence of the inflammatory symptoms in the oral cavity, generated by temporary problems occurring in individual objects, or generated by the presence of foreign substances.

Therefore, the score on the information on the color uniformity of the gingival area and the score on the information on the degree of recession of the same gum line as the corresponding tooth may be input to the feature fusion logic 2310, and the fusion score Z(f) may be output from the feature fusion logic 2310.

When the color uniformity of the gingival area and the degree of recession of the gum line of the same tooth as the gingival area are each greater than the predefined score and have the difference less than or equal to the reference value, the fusion score Z(f) of the corresponding tooth may be augmented according to the predefined method. The method in which the fusion score Z(f) of the tooth is augmented according to the predefined method may include a method of squaring the fusion score Z(f) of the tooth or a method of assigning a weighted score. The above weighted score may be a score that may not be derived using the color uniformity of the gingival area or the degree of recession of the gum line.

In addition, although not illustrated, the target of the feature fusion may be the information on the color uniformity of the crown area, the information on the color uniformity of the gingival area, and the information on the degree of recession of the gum line. The information on the color uniformity of the crown area, the information on the color uniformity of the gingival area, and the information on the degree of recession of the gum line are information that may affect the probability that the inflammation-related symptoms are present in the oral cavity.

Specifically, the further away the colors included in the targeted gingival area based on the RGB code, or the more similar the area with distant colors based on the included RGB code, or the more severe the degree of recession of the gum line, the higher the probability that the inflammation-related symptoms are present.

The fact that the colors included in one targeted crown area are far apart based on the RGB code may mean that tartar has accumulated on the crown, corrosion has occurred, cavities have occurred, or foreign substances are present on the crown. The fact that the colors included in one targeted gingival area are distant based on the RGB code may mean that the gingiva is swollen, bleeding has occurred in the gingiva, or foreign substances are present in the gingiva.

The fact that the areas with distant colors are similar based on the RGB code included in one targeted crown area may mean that tartar is excessively accumulated on the crown or that cavities have progressed significantly. The fact that the areas with distant colors are similar based on the RGB code included in one targeted gingival area may mean that the degree of swelling of the gingiva may be severe or excessive bleeding may have occurred in the gingiva.

The recession in one target gum line may be due to a physical accident occurring to the gum, leaving behind a scar. The gum line may differ depending on the oral structure of each individual.

As described above, the information affecting the color uniformity of the crown or gingival area may be generated by the presence of the inflammatory symptoms in the oral cavity and generated by temporary problems occurring in individual objects.

As described above, the information that affects the occurrence of recession in the gum line may be generated by the presence of the inflammatory symptoms in the oral cavity and generated by a physical accident that occurs in an individual object.

Therefore, the score on the information on the color uniformity of the gingival area, the score on the information on the color uniformity of the gingival area, and the score on the information on the degree of recession of the same gum line as the corresponding tooth may be input to the feature fusion logic 2310, and the fusion score Z(f) may be output from the feature fusion logic 2310.

When the color uniformity of the crown area, the color uniformity of the gingival area, and the degree of recession of the gum line of the same tooth as the gingival area are each greater than the predefined score and have the difference less than or equal to the reference value, the fusion score Z(f) of the corresponding tooth may be augmented according to the predefined method. The method in which the fusion score Z(f) of the tooth is augmented according to the predefined method may include the method of squaring the fusion score Z(f) of the tooth or the method of assigning a weighted score. The above weighted score may be a score that may not be derived using the color uniformity of the crown or gingival area or the degree of recession of the gum line.

The CNN 2110 that receives a captured image 2010 and outputs a score Z3 acquired by scoring the information on the degree of recession of the gum line of each segmented tooth will be described with reference to FIG. 56. The CNN 2110 that receives the captured image 2010 can output the information on the degree of recession of the gum lines of each individual tooth as the scoring score.

The information on the degree of recession is information that may be used to derive the degree of recession of the gum line. The degree of recession of the gum line refers to how far the shape and position of the gums recede compared to the teeth. The gum line is ideal if the gums surrounding the teeth form the same horizontal line as the teeth, but a tooth root between the tooth and the gums may recede in a direction in which it becomes exposed due to various factors. A typical factor that causes the recession of the gum line is periodontitis.

The CNN 2110 may output a result of scoring the degree of recession of the gum line of the targeted tooth using the curvature model of the gum lines of each individual tooth generated by performing the training with the normal dog image. The CNN 2110 may generate the curvature model of the gum lines of the individual tooth by performing the training with the image including the information on the curvature of the gum line. The curvature of the gum line may be the curvature of the line receding in the direction of the tooth root based on the same horizontal line as the tooth.

The CNN 2110 may output the result of scoring the degree of recession of the gum line of the targeted tooth using the operation model of the ratio of the first crown area among the first crown area generated by performing the training with the normal dog image and the entire first gingival area. The CNN 2110 may perform the training with the image including the information on the ratio of the first crown area among the first crown area and the entire first gingival area, and generate the operation model of the ratio of the first crown area among the first crown area and the entire first gingival area. In the operation model of the ratio of the first crown area among the first crown area and the entire first gingival area, the first crown area may be the area of the visible part of the targeted tooth, and the first gingival area may be the area of the gum part adjacent to the targeted tooth.

The recession progress of the gum line of one targeted tooth is determined by deriving the degree of curvature of an interface between the crown and the gingiva of the targeted tooth, and the degree of recession progress may be classified by deriving the ratio of the first crown area among the first crown area of the target tooth and the entire first gingival area.

Depending on the curvature of the gum line covering other teeth at each tooth location, it may be determined that the gum line of the tooth is receding. When it is determined that the gum line of the tooth is in the process of recession, the degree of recession of the gum line may be determined according to the area occupied by the gingival area of the target tooth in the area in which the gingival area and the crown area are combined. The curvature of the gum line is information that may affect the probability that the recession of the gum line has progressed, and the area occupied by the gingival area of the targeted tooth in the area in which the gingival area and the crown area are combined is information from which the degree of recession of the gum line can be determined.

The determination on the degree of recession based on the curvature of the gum line may be generated by a problem currently temporarily occurring in an individual object, and the determination on the degree of recession based on the area occupied by the gingival area in the area in which the gingival area and the crown area are combined may be a sign of injury to the gingival area.

In an image 2240 in which the score Z3 on the information on the degree of recession of the gum line of the tooth is overlaid on the captured image 2010, the more extreme the degree of recession of the gum line of the tooth, the higher a score may be output.

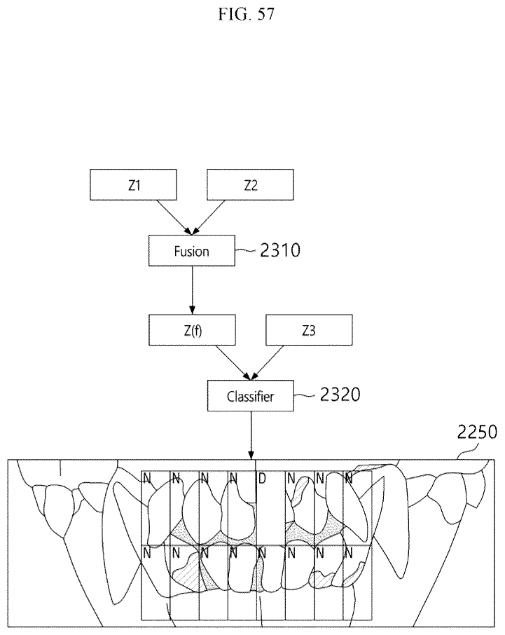

The architecture of the model of analysis of oral information will be described with reference to FIG. 57. According to this artificial neural network architecture, the CNN that receives the captured image 2010 outputs a feature score, the feature scores Z1 and Z2 are input to the fusion logic 2310, the fusion score Z(f) in the fusion logic is output, and the feature score Z3 and the fusion score Z(f) are input to the classifier 2320 for performing the classification based on the feature score.

An image 2250 in which information about the teeth at which periodontitis or inflammation is suspected is overlaid on the captured image may be output as a result of performing the classifier.

So far, the method of analysis of oral information in a pet according to some embodiments of the present disclosure has been described in detail. The embodiments described above are illustrative in all respects and should be understood as non-limiting.

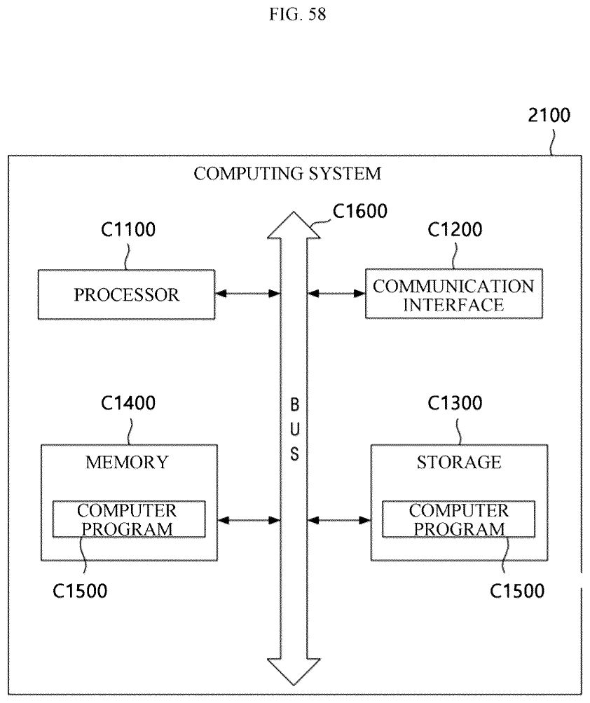

FIG. 58 is a hardware configuration diagram of a computing system according to some embodiments of the present disclosure. A computing system 2100 in FIG. 58 includes one or more processors C1100, a system bus C1600, a communication interface C1200, a memory C1400 into which the computer program C1500 executed by the processor C1100 is loaded, and a storage C1300 that stores the computer program C1500. For example, the computing system in FIG. 58 may be the system for assisting with analysis of oral information of a pet described with reference to FIG. 51.

The processor C1100 controls an overall operation of each component of the computing system 2100. The processor C1100 may perform operations on at least one application or program to execute methods/operations according to various embodiments of the present disclosure.

The memory C1400 stores various types of data, commands and/or information. The memory C1400 may load one or more computer programs C1500 from the storage C1300 to execute methods/operations according to various embodiments of the present invention. In addition, the memory C1400 may load a runtime for executing the model of analysis of oral information of a pet described with reference to FIGS. 3 to 7.

The definition data of the model of analysis of oral information of a pet may be data expressing the artificial neural network architecture that includes the CNN for outputting the first feature score using the information on the color uniformity of the first crown area and the information on the color uniformity of the first gingival area, the CNN for outputting the second feature score using the information on the degree of recession of the first gum line, and the classifier for performing the classification based on the first feature score and the second feature score

The bus C1600 provides a communication function between the components of the computing system 2100. The communication interface C1200 supports Internet communication of the computing system 2100. The storage C1300 may non-transitorily store one or more computer programs C1500. The computer program C1500 may include one or more instructions implementing methods/operations according to various embodiments of the present disclosure. When the computer program C1500 is loaded into the memory C1400, the processor C1100 may perform the methods/operations according to various embodiments of the present disclosure by executing the one or more instructions.

In some embodiments, the computing system 2100 described with reference to FIG. 58 may be configured using one or more physical servers included in a server farm based on cloud technology such as a virtual machine. In this case, at least some of the processor C1100, the memory C1400, and the storage C1300 among the components illustrated in FIG. 58 may be virtual hardware, and the communication interface C1200 may also be composed of virtualized networking elements such as a virtual switch.

The computer program C1500 may include an instruction to acquire a captured oral image of the pet, and instructions to input data of the acquired captured oral image to a model of analysis of oral information previously generated through machine learning, and generate an oral information analysis result using data output from the model of analysis of oral information, in which the model of analysis of oral information includes the artificial neural network that includes the CNN for outputting information on color uniformity of a first crown area, information on color uniformity of a first gingival area, and information on a degree of recession of a first gum line and a classifier for performing classification based on the information output from the CNN, the first gingival area is an area adjacent to the first crown area and showing a gingiva corresponding to a first crown expressed by the first crown area, and the first gum line is a boundary line between the first gingival area and the first crown area.

So far, various embodiments of the present disclosure and effects according to the embodiments have been described with reference to FIGS. 1 to 8. The effects according to the technical ideas of the present disclosure are not limited to the above-described effects, and other effects that are not mentioned may be obviously understood by those skilled in the art from the following description.

The technical ideas of the present disclosure described so far may be implemented as a computer-readable code on a computer-readable medium. The computer program recorded on the computer-readable recording medium may be transmitted to another computing device through a network such as the Internet and installed on the other computing device, and thus may be used on the other computing device.

According to a method and apparatus for determining patellar dislocation according to an embodiment of the present disclosure, it is possible to output dislocation information with high accuracy by calculating a diagnostic angle from a back view photo image of a pet.

In addition, according to a method and apparatus for determining patellar dislocation according to an embodiment of the present disclosure, it is possible to determine the presence or absence of patellar dislocation with high accuracy by removing a specific body area by pre-processing a back view image of a pet.

Although operations are illustrated in the drawings in a specific order, it should be understood that the operations do not need to be performed in the specific order illustrated or sequential order or that all illustrated operations should be performed to obtain the desired results. In specific situations, multitasking and parallel processing may be advantageous. Although embodiments of the present disclosure have been described with reference to the accompanying drawings, those skilled in the art will appreciate that various modifications and alterations may be made without departing from the spirit or essential feature of the present disclosure. Therefore, it is to be understood that the exemplary embodiments described hereinabove are illustrative rather than being restrictive in all aspects. The scope of the present disclosure should be interpreted from the following claims, and it should be interpreted that all the spirits equivalent to the following claims fall within the scope of the technical idea of the present disclosure.

What is claimed is:

1. A method of operating an apparatus for determining patellar dislocation that determines a joint disease in a pet, the method comprising:
an operation of obtaining a back view image including a hind leg area of the pet; and
an operation of outputting information on patellar dislocation in the pet using the back view image as an input to a pre-trained patellar dislocation prediction model,
wherein the patellar dislocation prediction model includes:
an operation of outputting first dislocation information of the pet when the back view image is a first back view image including first diagnostic angle information related to a patella of the pet; and
an operation of outputting second dislocation information of the pet when the back view image is a second back view image including information on a second diagnostic angle that is a different angle from the first diagnostic angle.

2. The method of claim 1, wherein the patellar dislocation prediction model includes an image classification model that outputs a feature vector from the back view image, and an object detection model that outputs a keypoint from the back view image.

3. The method of claim 2, wherein the patellar dislocation prediction model further includes a classifier that classifies the back view image based on the feature vector and the keypoint and outputs dislocation information of the pet.

4. The method of claim 1, wherein the dislocation information includes at least one of a probability of presence of dislocation, a degree of dislocation progression, and a dislocation position visualization image.

5. The method of claim 2, wherein the object detection model calculates a diagnostic angle for diagnosing the patellar dislocation based on the keypoint.

6. The method of claim 5, wherein the keypoint includes a first keypoint, a second keypoint, and a third keypoint, and
the first keypoint corresponds to an innermost point of a sole of one side of the pet, the second keypoint corresponds to an outermost point of a knee of one side of the pet, and the third keypoint corresponds to an uppermost point among points where a body of the pet intersects a line perpendicular to a ground and passing through the first point.

7. The method of claim 6, wherein the diagnostic angle is a Q angle calculated by a sum of a first angle formed by a first straight line that passes through the second keypoint and is perpendicular to the ground and a second straight line that passes through the first keypoint and the second keypoint and a second angle formed by the first straight line and a third straight line that passes through the second key point and the third key point.

8. The method of claim 5, wherein the keypoint is generated at left, right, or both sides of hind legs of the pet in the back view image.

9. The method of claim 8, wherein, when the keypoint is generated at both sides of hind legs of the pet, the classifier outputs the dislocation information of the pet using a Q angle of one hind leg having a wider Q angle among Q angles calculated for each of the both sides of hind legs of the pet as an input.

10. The method of claim 2, further comprising, before the outputting of the information on the patellar dislocation in the pet, an operation of performing pre-processing on the back view image.

11. The method of claim 10, wherein the operation of performing the pre-processing includes:
an operation of identifying a specific body area of the pet included in the back view image using an image classification model; and
an operation of removing a part or all of the identified specific body area from the back view image.

12. The method of claim 11, wherein the operation of performing the pre-processing further includes an operation of performing the pre-processing based on a breed of the pet,
removes the specific body area of the pet depending on whether the hind leg area is covered in the back view image when the pet included in the back view image is a first breed, and
does not remove the specific body area when the pet included in the back view image is a second breed.

13. The method of claim 12, wherein the operation of performing the pre-processing further includes an operation of outputting estimated keypoints from the back view image using the object detection model,
compares a position of at least one of the estimated keypoints with a position of the specific body area of the identified pet, and
determines that the hind leg area is covered to remove the specific body area of the pet when the position of the specific body area corresponds to a position of at least one of the estimated keypoints.

14. An apparatus for determining patellar dislocation, comprising:
a memory; and
at least one processor that executes instructions stored in the memory,
wherein the processor acquires a back view image including a hind leg area of a pet, and outputs information on patellar dislocation in the pet using the back view image as an input to a pre-trained patellar dislocation prediction model, and
the patellar dislocation prediction model outputs first dislocation information of the pet when the back view image is a first back view image including first diagnostic angle information related to a patella of the pet, and outputs second dislocation information of the pet when the back view image is a second back view image including information on a second diagnostic angle that is a different angle from the first diagnostic angle.

15. The method of claim 14, wherein the patellar dislocation prediction model includes an image classification model that outputs a feature vector from the back view image, and an object detection model that outputs a keypoint from the back view image.

16. The method of claim 15, wherein the patellar dislocation prediction model further includes a classifier that classifies the back view image based on the feature vector and the keypoint and outputs the dislocation information of the pet.

17. The method of claim 15, wherein, before outputting the information on the patellar dislocation in the pet, the processor performs pre-processing on the back view image.

18. The method of claim 17, wherein the processor identifies a specific body area of the pet included in the back view image using an image classification model, and removes a part or all of the identified specific body area from the back view image.

19. The method of claim 18, wherein the processor performs the pre-processing based on a breed of the pet, and
removes the specific body area of the pet depending on whether the hind leg area is covered in the back view image when the pet included in the back view image is a first breed, and does not remove the specific body area when the pet included in the back view image is a second breed.

20. The method of claim 19, wherein the processor outputs an estimated keypoint from the back view image using the object detection model, and
compares a position of at least one of the estimated keypoints with a position of a specific body area of the identified pet, and determines that the hind leg area is covered to remove the specific body area of the pet when the position of the specific body area corresponds to the position of at least one of the estimated keypoints.
